(19) United States
(12) Patent Application Publication
(10) Pub. No.: US 2003/0139715 A1
(43) Pub. Date: Jul. 24, 2003

(54) Absorbent materials having high stiffness and fast absorbency rates

(76) Inventors: Richard Norris Dodge II, Appleton, WI (US); Nancy Birbiglia Lange, Oshkosh, WI (US); Jayant Chakravarty, Appleton, WI (US); Jian Qin, Appleton, WI (US); Sridhar Ranganathan, Suwanee, GA (US); Fu-Jya Daniel Tsai, Appleton, WI (US); Cathleen M. Uttecht, Menasha, WI (US); Xiaomin Zhang, Appleton, WI (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/291,187

(22) Filed: Nov. 8, 2002

Related U.S. Application Data
(63) Continuation-in-part of application No. 10/017,683, filed on Dec. 14, 2001.

(51) Int. Cl. A61F 13/15; A61F 13/20

(52) U.S. Cl. 604/368

(57) ABSTRACT

Disclosed are absorbent composites, useful in an absorbent article, having a superabsorbent material having a high stiffness and a fast absorption rate. The superabsorbent materials of the absorbent composites of this invention have a stiffness index of greater than about 0.87 and a vortex time of less than about 40 seconds. Absorbent composites of this invention can have a high liquid intake rate and a rapid liquid lock-up. Absorbent composites of this invention can have an intake rate of at least about 1.9 cubic centimeter liquid/second at an 80% absorbent composite saturation level and a liquid lock-up fraction of at least about 0.70 at 50% superabsorbent material saturation. The absorbent composites of this invention can be a freeze-dried composite, an airformed absorbent composite, or other fibrous or non-fibrous absorbent composites.

### FIELD OF THE INVENTION

This invention relates to absorbent composites having improved multifunctional absorbent properties useful in absorbent articles. More specifically this invention relates to absorbent composites having superabsorbent materials having high stiffness and fast absorbency rates, and rapid fluid intake and rapid lock-up of liquid.

### BACKGROUND OF THE INVENTION

Various absorbent materials and structures are known in the art. Important characteristics of commercial absorbent materials and structures include either a high rate of fluid intake or rapid lock-up of liquid, but not both. Nonwoven surge materials, as taught in U.S. Pat. No. 5,490,846 to Ellis et al. and in U.S. Pat. No. 5,364,382 to Latimer, for example, have excellent intake functionality but typically almost no fluid retention properties. Current commercial diaper absorbent cores comprising an absorbent fluff and superabsorbent material combination typically provide good fluid absorbency but, often depending on the core density, typically poor fluid intake.

Increasing liquid intake rates in absorbent composites can be achieved through a variety of ways. Stiff superabsorbent particles, stiff fibers, and/or stabilization of the composite structure have been shown to be effective at achieving increased intake rate and sometimes maintaining that rate over multiple insults. However, swelling kinetics of the superabsorbent particles that dictate the speed of liquid lock-up into the superabsorbent particles are typically inadequate. Current commercial superabsorbent materials do not have the high stiffness properties as well as the fast absorbency rates of the superabsorbent materials of this invention.

A typical disposable absorbent product generally has a composite structure including a topsheet, a backsheet, and an absorbent structure between the topsheet and backsheet. In current commercial absorbent structures layers of different materials, such as a surge layer and an absorbent core layer, are required to provide desired fluid handling characteristics of high liquid intake and high liquid lock-up. The result may be an absorbent article with many production steps and high cost. There is a need for an absorbent composite having enhanced fluid intake and high liquid lock-up characteristics that could be used alone or in combination with other materials in an absorbent article.

### SUMMARY OF THE INVENTION

This invention is directed to absorbent composites having improved fluid handling properties and methods of making the absorbent composites. The absorbent composites of this invention include a superabsorbent material having high stiffness and a fast absorption rate, and can have a high intake rate of liquid and a high liquid lock-up fraction. Absorbent composites of this invention can include any foam, foam-like composite, airlaid composite, airformed composite, wetformed composite or combinations thereof. Absorbent composites of this invention can be modified using treatments such as ultraviolet radiation, ultrasonic, microwave radiation, and/or in-situ polymerization treatment to enhance liquid intake and lock-up performance.

In one embodiment of this invention the absorbent composite includes a superabsorbent material in combination with the fibers. The superabsorbent material has a stiffness index of greater than about 0.87, more suitably greater than about 0.92, and desirably greater than about 1.0, and a vortex time of less than about 40 seconds, more suitably less than about 30 seconds, and desirably about less than 20 seconds, as determined by test procedures described below. Superabsorbent materials having the disclosed stiffness index and vortex time can be newly manufactured or otherwise treated or modified to obtain these desired properties. The absorbent composite can include airformed, wet-formed or wetlaid, freeze-dried foam, or other absorbent composites known in the art.

In one embodiment of the invention, the absorbent composite includes a freeze-dried absorbent composite including a superabsorbent material having a stiffness index of greater than about 0.87 and a vortex time of less than about 40 seconds. The superabsorbent material of the freeze-dried absorbent composite has the desired stiffness index and vortex time after the freeze-dried composite has been formed, i.e. after the superabsorbent material has gone through the freeze-drying process to form the absorbent composite. The absorbent composite is made by forming a slurry comprising a water-insoluble fiber, and a binding agent. An absorbent material is then added to the slurry. The solution is cooled to a temperature between about −50° C. and 0° C. at a cooling rate effective to freeze the water. The frozen water is removed through sublimation and a fibrous absorbent composite is recovered. The freeze-dried composites of this invention desirably have an intake rate of at least about 1.9 cubic centimeters (cc) of liquid/second at 80% absorbent composite saturation and a liquid lock-up fraction of at least about 0.70 at 50% superabsorbent saturation, as determined by test procedures described below.

In one embodiment of this invention, the absorbent composite includes an airformed absorbent composite including a superabsorbent material having a stiffness index of greater than about 0.87 and a vortex time of less than about 40 seconds. The airformed composite is formed by mixing superabsorbent material and a fibrous material and using an airforming machine to lay down a web of intermingled fibers and superabsorbent materials onto a porous tissue. The airformed absorbent composites of this invention desirably have an intake rate of at least about 1.9 cubic centimeters liquid/second at 80% absorbent composite saturation and a liquid lock-up fraction of at least about 0.70 at 50% superabsorbent saturation.

In another embodiment of this invention, the absorbent composite is formed from a non-fibrous matrix including a superabsorbent material having a stiffness index of greater than about 0.87 and a vortex time of less than about 40 seconds. The non-fibrous absorbent composites of this invention desirably have an intake rate of at least about 1.9 cubic centimeters liquid/second at 80% absorbent composite saturation and a liquid lock-up fraction of at least about 0.70 at 50% superabsorbent saturation, as determined by test procedures described below.

Binding agents can be used in absorbent composites of this invention to provide strength to the absorbent composite structure both in the dry state and the wet state. Binding agents are water-insoluble in the absorbent composite and can bind the fibers of the absorbent composite together. Binding agents can be water-swellable and can be used to enhance liquid intake and liquid lock-up. A crosslinking agent may be needed to insolubilize a water-soluble binding agent after formation of the absorbent composite.

### BRIEF DESCRIPTION OF THE DRAWINGS

These and other objects and features of this invention will be better understood from the following detailed description taken in conjunction with the drawings, wherein:

FIG. 1 is an exploded perspective view of a diaper according to one embodiment of this invention.

FIG. 2 shows absorbent fibers in an absorbent structure according to one embodiment of this invention.

FIG. 3 is a photograph of an absorbent fibrous structure according to one embodiment of the invention.

FIG. 4A is a plan view of an intake rate testing device.

FIG. 4B is a top view of the intake rate testing device.

FIG. 5 is a perspective view of a liquid lock-up testing apparatus.

FIG. 6 is an illustration of equipment for determining the Absorbency Under Load (AUL) value of a superabsorbent material.

FIG. 7 is a cross-sectional view of the porous plate taken along line 7-7 of FIG. 6.

FIG. 8 is a plot of intake rate against composite saturation.

FIG. 9 is a plot of lock-up fraction against superabsorbent material saturation.

FIG. 10 is a plot of intake rate at 80% absorbent composite saturation against liquid lock-up fraction at 50% superabsorbent saturation.

### DESCRIPTION OF PREFERRED EMBODIMENTS

### Definitions

Within the context of this specification, each term or phrase below will include the following meaning or meanings.

“Foam” refers to two-phase gas-solid systems that have a supporting solid lattice of cell walls that are continuous throughout the structure. The gas, typically air, phase in a foam is usually distributed in void pockets often called cells. “Open-cell foams” are polymeric materials having substantial void space in the form of cells defined by a plurality of mutually connected, three dimensionally branched webs of polymeric material. The cells typically have openings to permit fluid communication from one cell to another. In other words, the individual cells of the foam are not completely isolated from each other by the polymeric material of the cell walls. The cells in such substantially open-celled foam structures have intercellular openings which are large enough to permit fluid transfer from one cell to another within the foam structure. For purposes of this invention, a foam material is “open-celled” if at least 50%, and desirably at least 80% of the cells in the foam structure that are at least about 1 micron size are in fluid communication with at least one adjacent cell.

“Superabsorbent saturation level” refers to the amount of liquid the superabsorbent material has absorbed as compared to, as a percentage, the total amount of liquid, or the total saturation level, the superabsorbent material is able to absorb. A 50% superabsorbent saturation level thus means that the superabsorbent material has absorbed 50% of the total amount of liquid the superabsorbent material is able to absorb.

“Absorbent composite saturation level” refers to the amount of liquid the absorbent composite (i.e. freeze-dried composite) has absorbed as compared to, as a percentage, the total amount of liquid, or the total saturation level, the absorbent composite is able to absorb. An 80% absorbent composite saturation level thus means that the absorbent composite has absorbed 80% of the total amount of liquid the absorbent composite is able to absorb

“Capillary size” refers to the size of the open cells in the fibrous composites of this invention. The capillaries, or interconnected open cells, are the passage ways through which fluids are taken into the absorbent fibrous composites.

“Hydrophilic” describes fibers or at least the surfaces of fibers which are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90° are designated “wettable” or hydrophilic, while fibers having contact angles greater than 90° are designated “nonwettable” or hydrophobic.

“Superabsorbent material” refers to a water-swellable, water-insoluble organic or inorganic material capable, under the most favorable conditions, of absorbing at least about 10 times its weight, preferably at least about 30 times its weight in an aqueous solution containing 0.9% by weight sodium chloride solution. Superabsorbent materials can comprise particles, fibers, and/or other structural forms. “Water-swellable, water-insoluble” refers to the ability of a material to swell to an equilibrium volume in excess water but not dissolve into the water. The water-swellable, water-insoluble material generally retains its original identity or physical structure, even in a highly expanded state during the absorption of water.

“Absorbent” refers to materials having an absorption capacity of greater than 5.0 grams of 0.9% by weight sodium chloride solution per gram of material.

“Non-absorbent” refers to materials having an absorption capacity of less than 5 times its weight in an aqueous solution containing 0.9% by weight sodium chloride solution.

“Water soluble” refers to materials which substantially dissolve in excess water to form a solution, thereby losing its initial form and becoming essentially molecularly dispersed throughout the water solution. As a general rule, a water-soluble material will be free from a substantial degree of crosslinking, as crosslinking tends to render a material water insoluble. A material that is “water insoluble” is one that is not water soluble according to this definition.

“Solvent” refers to a substance, particularly in liquid form, that is capable of dissolving a polymer used herein to form a substantially uniformly dispersed mixture at the molecular level.

The term “absorbent article” includes without limitation diapers, training pants, swim wear, absorbent underpants, baby wipes, adult incontinence products, feminine hygiene products, medical garments, underpads, bandages, absorbent drapes, and medical wipes, as well as industrial work wear garments.

The term “non-fibrous” includes, without limitation, absorbent structures containing no fibrous material, such as an open-celled foam.

These terms may be defined with additional language in the remaining portions of the specification.

This invention relates to absorbent composites having a fibrous or non-fibrous matrix and superabsorbent materials having a high stiffness and a fast absorption rate. “Stiffness” or “stiff” refers to the ability of the superabsorbent material to resist deformation against pressure while in a swollen state. “High stiffness” refers to a stiffness index of at least about 0.87 as determined by test procedures described below, and high stiffness superabsorbent materials are useful in the absorbent composites of this invention. The superabsorbent materials of this invention have a stiffness index of at least about 0.87, more suitably at least about 0.92, and desirably at least about 1.0. “Absorption rate” refers to ability of the superabsorbent material to rapidly absorb liquid. “Fast absorption rate” refers to superabsorbent materials useful in the absorbent composites of this invention. The superabsorbent materials of this invention having a fast absorption rate refers to superabsorbent materials having a vortex time of less than 40 seconds, more suitably less than about 30 seconds, and desirably less than about 20 seconds, as determined by test procedures described below. High stiffness and fast absorption rate superabsorbent materials are useful in absorbent composites for absorbent articles. Using a superabsorbent material with a high stiffness in an absorbent composite can provide a more open porous composite structure as the superabsorbent material does not deform as much during the swelling process (as compared to low stiffness superabsorbents).

High stiffness superabsorbent materials of one embodiment of this invention have a stiffness index of at least about 0.87, more suitably at least about 0.92, and more suitably at least about 1.0, and a vortex time of less than about 40 seconds. “Stiffness index” refers to the ratio of the absorbency under load (AUL) value of the superabsorbent material divided by the centrifuge retention capacity (CRC) value of the superabsorbent material. The absorbency under load of the superabsorbent material is determined by the Absorbency Under Load (AUL) Test described below at a load of 0.9 pounds per square inch. The centrifuge retention capacity of the superabsorbent material is determined by the Centrifuge Retention Capacity (CRC) Test also described below. “Vortex time” refers to the amount of time in seconds required for an amount of superabsorbent material to close a vortex created by stirring an amount of 0.9 percent (%) by weight sodium chloride solution according to the Vortex Time Test described below. The vortex times described herein are obtained at a superabsorbent material particle size range of about 300 to 600 microns. The superabsorbent materials of this invention have a vortex time of less than about 40 seconds, suitably less than about 30 seconds, and desirably less than about 20 seconds. Current commercial superabsorbent materials do not have a stiffness index of at least about 0.87 and a vortex time of less than about 40 seconds. Superabsorbent materials having a stiffness index of at least about 0.87 and a vortex time of less than about 40 seconds are useful in absorbent composites of this invention having a high intake rate and rapid lock-up of liquid.

The superabsorbent materials of this invention are useful in absorbent composites for use in absorbent articles such as a diaper. In one embodiment of this invention the absorbent article includes an absorbent composite that includes a superabsorbent material having a stiffness index of at least about 0.87 and a vortex time of less than about 40 seconds intermixed with water-insoluble fibers. The absorbent composites of this invention may be used alone or in combination with other absorbent or fluid handling layers', such as a surge layer. The absorbent composites are useful in absorbent articles such as diapers, training pants, swim wear, adult incontinence articles, feminine care products, and medical absorbent products.

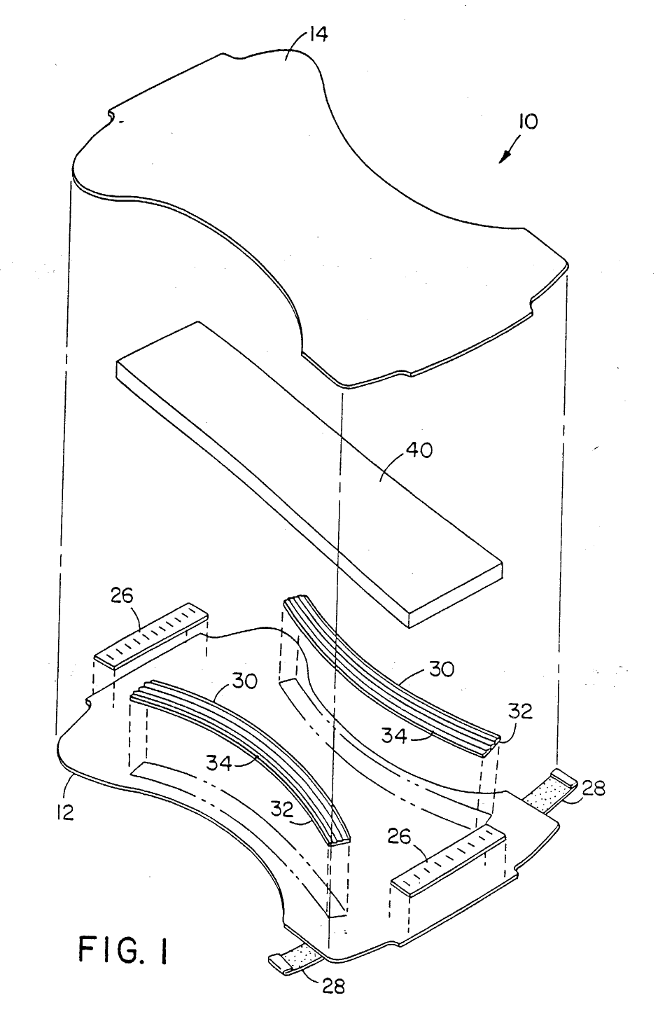

FIG. 1 illustrates an exploded perspective view of a typical disposable diaper. Referring to FIG. 1, disposable diaper 10 includes outer cover 12, body-side liner 14, and absorbent core 40 located between body-side liner 14 and outer cover 12. Absorbent core 40 can comprise any of the absorbent composites according to this invention. Outer cover 12 is constructed of conventional non-absorbent materials.

Body-side liner 14 is constructed from highly liquid pervious materials. This layer functions to transfer liquid from the wearer to the absorbent core 40. Suitable liquid pervious materials include porous woven materials, porous nonwoven materials, films with apertures, open-celled foams, and batting. Examples include, without limitation, any flexible porous sheets of polyolefin fibers, such as polypropylene, polyethylene, or polyester fibers; webs of spunbonded polypropylene, polyethylene, or polyester fibers; webs of rayon fibers; bonded carded webs of synthetic or natural fibers; or combinations thereof. The various layers of article 10 have dimensions which vary depending on the size and shape of the wearer.

Attached to outer cover 12 may be waist elastics 26, fastening 28, and leg elastics 30. The leg elastics 30 typically have a carrier sheet 32 and individual elastic strands 34. The diaper of FIG. 1 is a general representation of one basic diaper embodiment. Various modifications can be made to the design and materials of diaper parts. For example, a surge material can be placed between the body-side liner 14 and the absorbent core 40, or placed between the absorbent core 40 and the outer cover 12. Surge material is typically a non-absorbent nonwoven material which has a high intake of fluid and is useful in temporarily storing and distributing fluids to the absorbent material.

As will be appreciated by one skilled in the art reading this patent application, the absorbent composites of this invention can be used with various absorbent articles including various additional configurations and materials. Possible construction methods and materials of an embodiment of a diaper such as illustrated in FIG. 1, are set forth in greater detail in commonly assigned U.S. Pat. No. 5,509,915, issued Apr. 23, 1996 in the name of Hanson et al., incorporated herein by reference. Possible modifications to the diaper illustrated in FIG. 1 are set forth in commonly assigned U.S. Pat. No. 5,509,915 and in commonly assigned U.S. Pat. No. 5,364,382, issued Nov. 15, 1994 to Latimer et al.

The absorbent composites of this invention include one or more superabsorbent materials of this invention having a high stiffness and fast absorption rate, and can be formed as a freeze-dried composite, an airformed absorbent composite, wetformed absorbent composite, or other absorbent fibrous or non-fibrous composites. These composites comprise a water-swellable, water-insoluble superabsorbent material and an insoluble fiber. The absorbent composites of this invention can also include non-fibrous absorbent composites. The absorbent composites of this invention can also include one or more superabsorbent materials. Any superabsorbent material having a stiffness index of at least about 0.87 and a vortex time of less than about 40 seconds can be used in the absorbent composites and absorbent articles of this invention. The physical form of the superabsorbent material can include particulate, fibrous, nonwoven aggregate, printed, coated, or other forms.

The high stiffness superabsorbent materials of this invention can provide an open, porous absorbent composite structure, allowing for rapid liquid intake. The open, porous structure can also be influenced by the composite forming process and the nature of the fibers as well as the superabsorbent stiffness. In one embodiment of this invention the fibers have a high stiffness. Fiber “stiffness” refers to the ability of the fiber to resist bending and deformation while in a wetted state. Using a fiber with a high stiffness can also provide a more open, porous absorbent composite structure because the fiber does not bend and deform as much in the wetted state as do low stiffness fibers. Examples of high stiffness fibers include fibers available from Buckeye Corporation, Memphis, Tenn., designated as CARESSA® 1300, and from Weyerhauser Corporation, Federal Way, Washington, designated as NHB-416. Absorbent composite forming processes can also promote a more open, porous structure. Forming processes can cause the components of the composite to interact in such a way as to cause the structure to maintain an open, porous structure in the wetted state and after the superabsorbent is swollen. Such forming processes include but are not limited to freeze-drying, wet-forming, and airforming. Open structures can allow rapid intake of fluid into the composite.

In one embodiment of this invention, the absorbent composite includes a superabsorbent material having a stiffness index of at least about 0.87 and a vortex time of less than about 40 seconds, and has both a high liquid intake rate, and a rapid lock-up of liquid. “Intake rate” refers to the volume of liquid that is transferred into a composite as a function of time, as determined by the Intake Rate Test procedure described below. “High intake rate” refers to an intake rate of at least about 1.9 cubic centimeters liquid/second at 80 percent absorbent composite saturation. The liquid simply must enter into the composite and may be present as free liquid in the interstitial space of the absorbent composite, as liquid absorbed into a superabsorbent material, and liquid that has passed through the composite.

“Lock-up” refers to the amount of liquid absorbed into the superabsorbent material of the absorbent composite within a predetermined amount of time, as determined by the Liquid Lock-up Test procedure described below. “Rapid lock-up” refers to a liquid lock-up fraction, as determined by the Liquid Lock-up Test procedure described below, of at least about 0.70 at 50 percent superabsorbent saturation. A high intake rate, in particular, results in substantial leakage control. The combined benefits of a high intake rate and rapid lock-up fraction result in an absorbent composite which can quickly contain a liquid insult and prevent that liquid from being expelled from the composite under pressure or gravity. Superabsorbent materials having a stiffness index of at least about 0.87 and a vortex time of less than about 40 seconds can be used to provide an absorbent composite with the desired high intake rate and rapid liquid lock-up fraction of this invention.

The absorbent composites of one embodiment of this invention exhibit high intake rates and rapid lock-up of liquid. The high intake rates of the absorbent composites of this invention are defined at the 80% composite saturation level. The rapid liquid lock-up properties of the absorbent composites of this invention are defined as a fraction of the amount of liquid absorbed by the superabsorbent material over the total added liquid to the absorbent composite at a 50% superabsorbent material saturation level. To determine the intake rate and lock-up fraction of an absorbent composite at these saturation levels it may be necessary to obtain data at various saturation levels and interpolate the intake rate and lock-up fraction at the 80% composite saturation and 50% superabsorbent material saturation levels. These particular saturation levels are important when absorbent composites are used within absorbent articles. Absorbent composites generally have difficulty maintaining intake rate at higher saturation levels, and thus the high saturation levels are where leakage of body fluids from absorbent articles typically occurs. Likewise, the choice of 50% superabsorbent material saturation level as the point to characterize liquid lock-up behavior of this invention is due to the fact that the swelling kinetics of superabsorbent materials typically become relatively slow at these higher swelling levels, thus diminishing the ability to rapidly lock-up liquid. The diminishing ability to lock-up liquid typically results in a tendency for absorbent products to leak.

Absorbent composites according to one embodiment of this invention have an intake rate of at least about 1.9 cubic centimeters liquid/second at 80% composite saturation level and a liquid lock-up fraction of at least about 0.70, more suitably at least about 0.75, and desirably at least about 0.80, at 50% superabsorbent material saturation level. Alternatively, absorbent composites according to another embodiment of this invention have an intake rate of at least about 2.3 cubic centimeters liquid/second at 80% composite saturation level and a liquid lock-up fraction of at least about 0.70 at 50% superabsorbent material saturation level. Absorbent composites according to another embodiment of this invention have an intake rate of at least about 2.7 cubic centimeters liquid/second at 80% composite saturation level and a liquid lock-up fraction of at least about 0.80 at 50% superabsorbent material saturation level. Absorbent composites according to yet another embodiment of this invention have an intake rate of at least about 3.3 cubic centimeters liquid/second at 80% composite saturation level and a liquid lock-up fraction of at least about 0.80 at 50% superabsorbent material saturation level.

The intake rates and lock-up fractions of absorbent composites of this invention can be dependent on the specific combination of three characteristics: 1) the structure of the composite (freeze-dried composite, airformed, etc.); 2) the superabsorbent material used; and 3) the fiber type used. As will be seen by the examples below, making two airformed composites from the same superabsorbent material having a high stiffness and fast absorption rate and different fiber type results in one composite having the desirable intake rate and liquid lock-up of this invention and one that does not.

In one embodiment of this invention the absorbent composite is a freeze-dried absorbent composite including a superabsorbent material having a stiffness index of at least about 0.87 and a vortex time of less than about 40 seconds. The superabsorbent material of the freeze-dried absorbent composite has the stiffness index and vortex time of this invention after the freeze-dried composite has been formed, i.e. after the superabsorbent material has gone through the absorbent composite freeze-drying process. The method of making the freeze-dried absorbent composite according to one embodiment of this invention includes forming a slurry of water, a binding material, and a water insoluble fiber material. A water-swellable, water-insoluble superabsorbent material is then added to the slurry and the slurry is cooled to a temperature appropriate to freeze the water. The water is removed from the slurry under a high vacuum while the slurry is still in the frozen state, and a freeze-dried absorbent composite is recovered. Co-pending U.S. Patent Application filed on Dec. 14, 2001, having Ser. No. 10/017,465, discloses embodiments of freeze-dried composites and methods for making freeze-dried composites that can be used to make the freeze-dried composites of this invention, and is herein incorporated by reference.

Suitable freezing temperature for making a freeze-dried fibrous composite is below the freezing point of the slurry solvent used. When water is used as the slurry solvent the temperature should be about 0° C. to about −50° C., suitably from about −5° C. to −50° C., more suitably from about −10° C. to −40° C., and desirably from about −10° C. to −30° C. The selection of temperature is also dependent on the nature and concentration of the slurry. If the temperature selection is too close to the freezing point of the polymer slurry solution the frozen slurry may not have enough strength and may deform under vacuum removal of the solvent. If the temperature drops too far below the solvent freezing point the solvent molecules may form crystals which generally cause substantial cracks in the composite and reduce mechanical properties of the recovered composites.

While freezing the slurry it is important to control the cooling rate of the slurry from room temperature (˜23° C.) to freezing temperature. The cooling rate should not exceed a critical cooling rate. “Critical cooling rate” refers to the cooling rate at which, or any rate greater, the slurry, as well as the final absorbent composite, begins to form visible cracks or visible non-uniformity. The critical cooling rate can vary depending upon the freezing point of the solvent used, the slurry concentration, use of a two solvent slurry, the solvent crystallizability, the ratio of insoluble fibers to superabsorbent material, and the ratio of fibers to binding agent. A cooling rate slower than the critical cooling rate is preferred and generally results in a much more uniform pore structure and a softer, more flexible absorbent composite, due to the elimination of substantial cracks caused by uneven crystallization of solvent molecules. The cooling rate for an aqueous slurry having a weight ratio of insoluble fibers to soluble polymer greater than 9:1 or a weight ratio of water-swellable superabsorbent material to water-soluble polymer greater than 9:1, should be from about 0.01° C. to 10° C. per minute, more suitably from about 0.05° C. to 3° C. per minute, and desirably from about 0.1° C. to 1° C. per minute.

Removal of the frozen solvent is preferably done by vacuum sublimation. Vacuum suitable for this process is dependent on the volatility of the solvent used. Higher vacuum can increase the rate of sublimation and lower vacuum applies a lower pressure on the frozen slurry that can result in less damage and a higher mechanical strength of the resulting composite. Vacuum conditions are desirably less than about 500 millitorrs, or less than about 300 millitorrs, or less than 200 millitorrs, or less than 100 millitorrs. In general, good vacuum can be achieved by either a good quality vacuum pump or a lower condenser temperature, which captures more water vapor. Because sublimation is endothermic, the temperature of the frozen slurry is reduced as water is sublimated under vacuum. This means that the frozen slurry will be even colder and therefore it becomes more difficult to release water molecules. In order to compensate such energy loss, the freeze dryer should be equipped with a heater which provides just enough heat to compensate the energy loss to maintain temperature at a predetermined level.

The freeze-dried composites of this invention desirably have an intake rate of at least about 1.9 cubic centimeters liquid/second at 80% freeze-dried composite saturation and a liquid lock-up fraction of at least about 0.70, more suitably at least about 0.75, and desirably at least about 0.80, at 50% superabsorbent material saturation. Alternatively, the freeze-dried composites of this invention have an intake rate of at least about 2.3 cubic centimeters liquid/second at 80% freeze-dried composite saturation and a liquid lock-up fraction of at least about 0.70 at 50% superabsorbent material saturation. As another alternative, the freeze-dried composites of this invention have an intake rate of at least about 2.7 cubic centimeters liquid/second at 80% freeze-dried composite saturation and a liquid lock-up fraction of at least about 0.80 at 50% superabsorbent material saturation. As yet another alternative, the freeze-dried composites of this invention have an intake rate of at least about 3.3 cubic centimeters liquid/second at 80% freeze-dried composite saturation and a liquid lock-up fraction of at least about 0.80 at 50% superabsorbent material saturation.

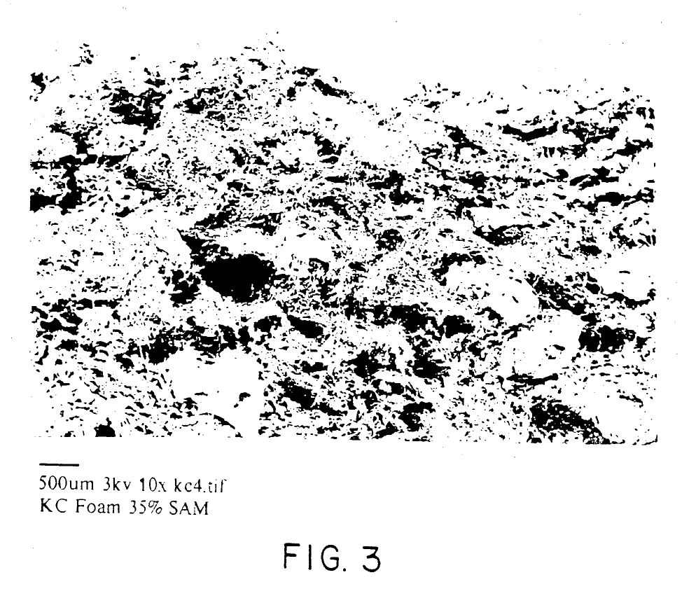

The resulting freeze-dried composite, as shown in FIG. 3, is a soft freeze-dried absorbent composite including a water-swellable, water-insoluble superabsorbent polymer and a water insoluble fiber. The water-swellable, water-insoluble superabsorbent material is present in the absorbent composite in an amount of at least about 10% by weight, suitably from about 10% to 70% by weight, alternatively from about 20% to 60% by weight, or from about 30% to 50% by weight. The water-insoluble fiber is present in the absorbent composite in an amount from about 30% to 90% by weight, suitably from about 40% to 80% by weight, or from about 50% to 70% by weight. The binding material is present in the absorbent composite in an amount from about 0% to 20% by weight, suitably from about 1% to 10% by weight, and alternatively from about 2% to 8% by weight.

Absorbent composites of another embodiment of this invention include airformed absorbent composites. Airformed absorbent composites are made by combining superabsorbent particles and matrix fibers into an airforming former unit to mix and lay down a web of intermingled superabsorbent particles and matrix fibers. The web of intermingled superabsorbent particles and matrix fibers is formed directly onto a porous sheet of tissue. One example of a suitable porous tissue is designated as 9.8 pound White Forming Tissue available from American Tissue, Inc., Neenah, Wis. The airformed absorbent composite can then be compressed to a desired density by a Carver Press. A suitable airformed absorbent composite density is between about 0.05 grams/cubic centimeters (g/cc) and 0.5 grams/cubic centimeters, a more suitable density is between about 0.1 grams/cubic centimeters and 0.4 grams/cubic centimeters, and a preferred density is between about 0.15 grams/cubic centimeters and 0.35 grams/cubic centimeters. The water-swellable, water-insoluble superabsorbent material is present in the absorbent composite in an amount of at least about 10% by weight, suitably from about 10% to 70% by weight, desirably from about 20% to 60% by weight, and preferably from about 30% to 50% by weight. The water-insoluble fiber is present in the absorbent composite in an amount from about 30% to 90% by weight, suitably from about 40% to 80% by weight, and desirably about 50% to 70% by weight. A binding material may be present in the absorbent composite in an amount from about 0% to 20% by weight, suitably from about 1% to 10% by weight, and alternatively from about 2% to 8% by weight.

Absorbent composites of another embodiment of this invention include wetformed absorbent composites. Wetformed absorbent composites are formed by processes well known in the art. One example of a process for making wetformed absorbent composite is disclosed in U.S. Pat. No. 5,651,862, issued to Anderson et al. on Jul. 29, 1997, herein incorporated by reference. Wetformed composites are generally formed by mixing fibers, absorbent materials, and other possible additives such as binder materials into a liquid medium. The medium plus the fibers, absorbent materials, and the other possible additives are conveyed onto a web forming porous substrate, and the medium is removed by vacuum. The water-swellable, water-insoluble superabsorbent material is present in the absorbent composite in a weight amount of at least about 10% by weight, suitably about 10% to 70% by weight, desirably about 20% to 60% by weight, and preferably about 30% to 50% by weight. The water-insoluble fiber is present in the absorbent composite in a weight amount from about 30% to 90% by weight, suitably about 40% to 80% by weight, and desirably about 50% to 70% by weight.

Water-insoluble fibers suitable for the various absorbent composites of this invention include both natural fibers including without limitation, wood pulp, cotton linter, synthetic fibers including without limitation, thermoplastic fibers such as polyethylene fibers, polypropylene fibers, poly(ethylene terephthalate) fibers, polyester fibers, and elastic fibers such as polyurethane fibers. Hydrophilic fibers are preferred due to their wettability characteristics. Hydrophobic fibers can be used and are preferably treated with surfactants or other effective treatment to alter surface chemistry to increase wettability.

Fiber size can directly affect capillary structure of the final absorbent composite. Generally, the larger the fiber size, the larger the capillary size, and when the capillary size gets larger, the ability to move liquid to high heights against gravity is diminished. Oppositely, smaller fiber size generally provides smaller capillary size which can move liquid up to high heights against gravity, but the rate of liquid movement may be negatively impacted. Therefore, an appropriate fiber size choice is critical based on the final function desired. Generally, larger and stiffer fibers are preferred because they lead to structures with larger capillaries that would improve the intake rate of the composite. Fibers useful in this invention have a diameter of about 1 micron to 100 microns, suitably about 1 micron to 50 microns, and desirably about 10 microns to 30 microns.

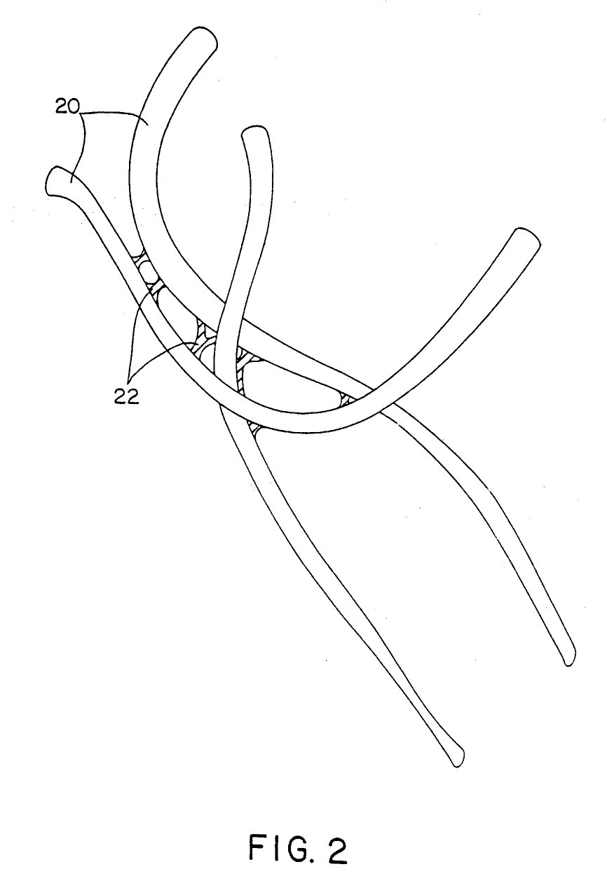

Binding materials provide strength to the absorbent fibrous composite both in the dry state and the wet state. Binding materials can be water-soluble initially and water-insoluble in the absorbent fibrous composite after heat curing. Binding materials bind the water-insoluble fibers and the superabsorbent materials together. As shown in FIG. 2, fibers 20 are held together in the fibrous absorbent composite structure by binding material polymers 22. The binding material may be water-swellable or not water-swellable. When used in absorbent articles, the binding material is desirably water-swellable. Desirable binding material polymers are hydrophilic and substantially water-insoluble in the absorbent fibrous composite, providing desired wet strength of the fibrous composite.

For swellable binding materials, high molecular weight ionic polymers such as sodium-polyacrylate, carboxymethyl cellulose, and chitosan salt are useful in that they provide strength and absorbency to the freeze-dried composite. Other swellable binding materials include isobutylene-maleic anhydride copolymers, polyvinyl amines, polyquarternary ammoniums, polyvinyl alcohols, hydroxypropyl celluloses, polyethylene oxides, polypropylene oxides, polyethylene glycols, modified polysaccharides, proteins, and combinations thereof. Non-swellable, low molecular weight binding materials include poly(aminoamide) epichlorohydrin polymer, such as KYMENE® (available from Hercules Inc., Chicopee, Mass.), latex, and other adhesives. Other non-swellable binding materials include any wet strength resins used in the paper making industries and any type of adhesive material. If an adhesive is used it is preferred that the adhesive is hydrophilic.

A crosslinking agent may be needed to insolubilize a water-soluble binding material after formation of the absorbent composite structure. Crosslinking agents are typically water-soluble. Suitable crosslinking agents include organic compounds comprising at least two functional groups capable of reacting with at least one of carboxyl, carboxylic acid, amino, and/or hydroxyl groups. Examples of this type of crosslinking agents include without limitation, diamines, polyamines, diols, polyols, polycarboxylic acids, and polyoxides. Another suitable crosslinking agent is a metal ion with more than two positive charges, including without limitation, Al³⁺, Fe³⁺, Ce³⁺, Ce⁴⁺, Ti⁴⁺, Zr⁴⁺, and Cr³⁺. When cationic polymer binding agents are used, polyanionic substances are suitable crosslinking agents. Polyanionic substances include without limitation, sodium-polyacrylate, carboxymethyl cellulose, and polymers including the phosphate anion —PO₄³⁻.

In one embodiment of this invention, the method of making a freeze-dried absorbent composite includes forming a slurry of water and a water insoluble fiber material with no binding material added to the slurry. A water-swellable, water-soluble superabsorbent precursor is added to the slurry and the slurry is cooled to a temperature appropriate to freeze the water. The water is removed from the slurry under high vacuum sublimation, and an absorbent composite is recovered.

When the water-soluble superabsorbent precursor is used, there is no need to add a binding material. The superabsorbent material will act as the binding material when the water-soluble superabsorbent precursor is crosslinked to form a water-swellable, water-insoluble network after composite production. The resulting composite is also a soft absorbent composite comprising a water-swellable, water-insoluble superabsorbent polymer and a water insoluble fiber. However, the superabsorbent materials for use in this invention have a stiffness index of at least about 0.87 and a vortex time of less than about 40 seconds. The stiffness index and the vortex time of superabsorbent materials in absorbent composites crosslinked from water-soluble superabsorbent precursors can be tested by crosslinking equivalent superabsorbent precursors not formed in a composite.

When using a superabsorbent precursor or a water-soluble binding agent in this invention, a crosslinking agent is added. Subsequently, the absorbent composite may require treatment to induce the crosslinking to provide a water-insoluble superabsorbent material or a water-insoluble binding agent. Suitable post composite treatment includes without limitation, heat curing at temperature greater than 60° C., ultraviolet radiation, microwave radiation, steam or high pressure, electronic beam radiation, organic solvents, and humidity treatment.

In the various embodiments of this invention, many suitable types of wettable, hydrophilic fibrous materials can be used to form the absorbent composites. Suitable matrix fibers include without limitation, naturally occurring organic fibers composed of inherently wettable material, such as cellulose fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers; and synthetic fibers composed of a nonwettable thermoplastic polymer, such as polypropylene fibers. The fibers may be hydrophilized, for example, by treatment with silica, treatment with a material which has a suitable hydrophilic moiety and is not readily removable from the fiber, or by sheathing the nonwettable, hydrophobic fiber with a hydrophilic polymer during or after the formation of the fiber. Combinations of various fibers can also be used in absorbent composites of this invention.

Other embodiments of this invention may include a non-fibrous matrix. One example of a non-fibrous matrix is an open-celled foam. Absorbent composites including a non-fibrous matrix can be formed by mixing superabsorbent and the non-fibrous matrix in such a way as to distribute the superabsorbent uniformly or non-uniformly throughout the non-fibrous matrix. The water-swellable, water-insoluble superabsorbent material may be present in the absorbent composite in an amount of at least about 10% by weight, suitably about 10% to 70% by weight, desirably about 20% to 60% by weight, and preferably about 30% to 50% by weight. The non-fibrous matrix may be present in the absorbent composite in an amount about 30% to 90% by weight, suitably about 40% to 80% by weight, and desirably about 50% to 70% by weight. A binding material may be present in the absorbent composite in an amount from about 0% to 20% by weight, suitably from about 1% to 10% by weight, and alternatively from about 2% to 8% by weight.

Known superabsorbent materials include, without limitation, (1) the anionic polymers, such as the alkali metal and ammonium salts of poly(acrylic acid), poly(methacrylic acid), isobutylene-maleic anhydride copolymers, poly(vinyl acetic acid), poly(vinyl phosphonic acid), poly(vinyl sulfonic acid), carboxymethyl cellulose, carboxymethyl starch, carrageenan, alginic acid, polyaspartic acid, polyglutamic acid, and combinations and copolymers thereof; (2) the cationic polymers, such as salts of poly(vinyl amine), poly(ethylene imine), poly(amino propanol vinyl ether), poly(allyl amine), poly(quaternary ammonium), poly(diallyl dimethyl ammonium hydroxide), polyasparagins, polyglutamines, polylysines, polyarginines, and combinations and copolymers thereof, (3) the mixture of anionic and cationic superabsorbent polymers, such as any combination of at least each one from Groups (1) and (2); (4) the mixture of acidic and basic polymers, such as acidic polymers from non-neutralized anionic superabsorbent polymers of Group (1) and basic polymers from non-neutralized cationic superabsorbent polymers of Group (2). The absorbent composites of this invention can include superabsorbent materials such as a crosslinked anionic polymer and a crosslinked cationic polymer, and can include superabsorbent polymers including monomers present in current commercial superabsorbent materials. However, the present invention relates, in at least one aspect, to superabsorbent materials having a stiffness index of at least about 0.87 and a vortex time of less than about 40 seconds, as well as the use of such materials in the proper combination to allow formation of the improved absorbent composites and absorbent articles described herein.

It has been discovered that current commercial superabsorbent materials not having a stiffness index of at least about 0.87 and a vortex time of less than about 40 seconds can be modified to obtain these desired properties after modification. One such method of obtaining a superabsorbent material having a stiffness index of at least about 0.87 and a vortex time of less than about 40 seconds is to use freeze-drying techniques disclosed in co-pending U.S. Patent Application filed by Express Mail No. EV068478187US on Nov. 8, 2002, and having Ser. No. ______, herein incorporated by reference, and similar to those described above. One example of such a modified commercial superabsorbent material is a modified freeze-dried superabsorbent material designated FAVOR® SXM 9543, available in unmodified form from Stockhausen, Inc., Greensboro, N.C. Freeze-dried superabsorbent materials can be obtained by absorbing an amount of water or other solvent, freezing the swollen superabsorbent material, and removing the water by sublimation using a freeze-dryer or similar device. In one embodiment of this invention, the superabsorbent material has a stiffness index of greater than about 0.87 and a vortex time of greater than about 40 seconds before freeze-drying, and a stiffness index of greater than about 0.87 and a vortex time of less than about 40 seconds before freeze-drying. One specific method used to freeze-dry FAVOR® SXM 9543 is described below.

According to one embodiment of this invention, the airformed, wetformed, freeze-dried foams, and non-fibrous absorbent composites include a superabsorbent material having a stiffness index of at least about 0.87 and a vortex time of less than about 40 seconds. The airformed, wetformed, and non-fibrous absorbent composites of this invention desirably have an intake rate of at least about 1.9 cubic centimeters liquid/second at 80% absorbent composite saturation and a liquid lock-up fraction of at least about 0.70, more suitably at least about 0.75, and desirably at least about 0.80, at 50% superabsorbent material saturation. Alternatively, the airformed, wetformed, and non-fibrous absorbent composites of this invention have an intake rate of at least about 2.3 cubic centimeters liquid/second at 80% absorbent composite saturation and a liquid lock-up fraction of at least about 0.70 at 50% superabsorbent material saturation. As another alternative, the airformed, wetformed, and non-fibrous absorbent composites of this invention have an intake rate of at least about 2.7 cubic centimeters liquid/second at 80% absorbent composite saturation and a liquid lock-up fraction of at least about 0.80 at 50% superabsorbent material saturation. The airformed, wetformed, and non-fibrous absorbent composites of yet another embodiment of this invention have an intake rate of at least about 3.3 cubic centimeters liquid/second at 80% absorbent composite saturation and a liquid lock-up fraction of at least about 0.80 at 50% superabsorbent material saturation.

Absorbency Under Load (AUL) Test

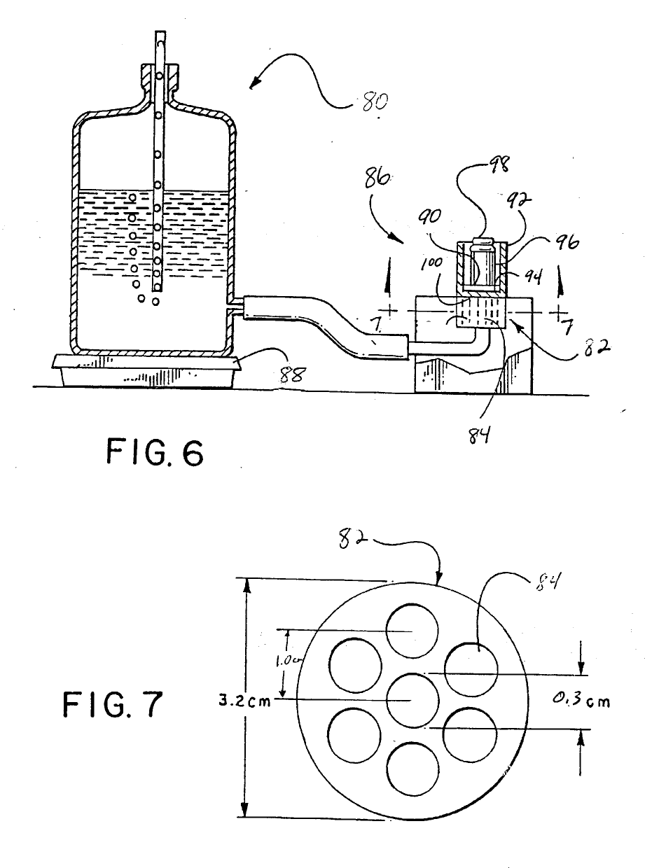

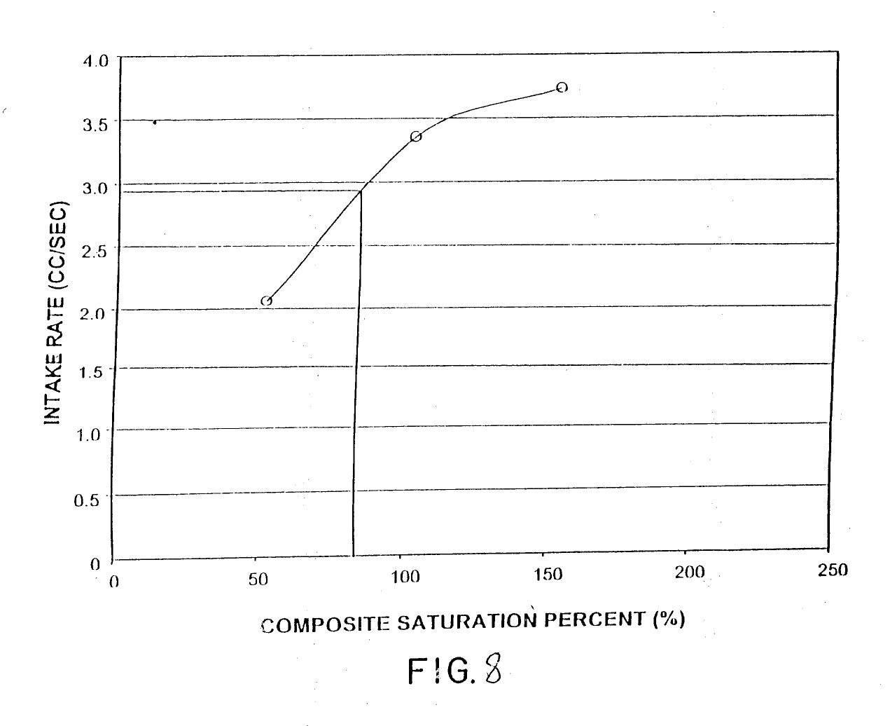

The Absorbency Under Load (AUL) test is a measure of the ability of a superabsorbent material to absorb a liquid while the superabsorbent material is under a restraining load. The test may best be understood by reference to FIGS. 6 and 7. Referring to FIG. 6, a demand absorbency tester (DAT) 80 is used, which is similar to a GATS (gravimetric absorbency test system), available from M/K Systems, Danners, Mass., as well as a system described by Lichstein in pages 129-142 of the INDA Technological Symposium Proceedings, March 1974.

A porous plate 82 is used having ports 84 confined within the 2.5 centimeters diameter covered, in use, by the Absorbency Under Load apparatus 86. FIG. 7 shows a cross-sectional view of porous plate 82. The porous plate 82 has a diameter of 3.2 centimeters with 7 ports (holes) 84 each with diameter of 0.30 centimeters. The porous plate 82 has one hole 84 in the center and the holes are spaced such that the distance from the center of one hole to another adjacent to it is 1.0 centimeter. An electrobalance 88 is used to measure the flow of the test fluid (an aqueous solution containing 0.9% by weight sodium chloride) into the superabsorbent material 90.

The AUL apparatus 86 used to contain the superabsorbent material may be made from 1 inch (2.54 centimeters), inside diameter, thermoplastic tubing 92 machined-out slightly to be sure of concentricity. A #100 mesh stainless steel wire cloth 94 is adhesively attached to the bottom of tubing 92. Alternatively, the steel wire cloth 94 may be heated in a flame until red hot, after which the tubing 92 is held onto the cloth until cooled. Care should be taken to maintain a flat, smooth bottom and not distort the inside of the tubing 92. A 4.4 gram piston 96 may be made from 1 inch (2.54 centimeters) solid material (e.g., Plexiglas) and machined to closely fit, without binding, in the tubing 92. A 317 gram weight 98 is used to provide 62,000 dynes per square centimeter (about 0.9 pounds per square inch (psi)) restraining load on the superabsorbent material. For the purpose of the present invention, the pressure applied during the AUL test is 0.9 psi.

Desirably, about 0.160 grams of superabsorbent is used. The sample is taken from superabsorbent material, which is pre-screened through U.S. standard #30 mesh screen and retained on U.S. standard #50 mesh screen. The superabsorbent material, therefore, has a particle size of about 300 to 600 microns. The particles may be pre-screened by hand or automatically pre-screened with, for example, a Ro-Tap Mechanical Sieve Shaker Model B available from W. S. Tyler, Inc., Mentor, Ohio.

The desired amount of superabsorbent material 90 (0.160 grams) is weighed onto weigh paper and placed on the wire cloth 94 at the bottom of the tubing 92. The tubing 92 is shaken to level the superabsorbent material on the wire cloth 94. Care is taken to be sure no superabsorbent material is clinging to the wall of the tubing 92. The piston 96 and weight 98 are carefully placed on the superabsorbent material to be tested. The test is initiated by placing a 3 centimeter diameter glass filter paper 100 (Whatman filter paper Grade GF/A, available from Whatman International Ltd., Maidstone, England) onto the plate 82 (the paper is sized to be larger than the internal diameter and smaller than the outside diameter of the tubing 92) to ensure good contact, while eliminating evaporation over the ports 84 of the demand absorbency tester 80 and then allowing saturation to occur. The device is started by placing the apparatus 86 on the glass filter paper 100 and allowing saturation to occur. The amount of fluid picked up is monitored as a function of time either directly by hand, with a strip chart recorder, or directly into a data acquisition or personal computer system.

The amount of fluid pick-up measured after 60 minutes is the AUL value and is reported in grams of test liquid absorbed per gram of superabsorbent material as determined before starting the test procedure. A check can be made to ensure the accuracy of the test. The apparatus 86 can be weighed before and after the test with a difference in weight equaling the fluid pick-up.

Centrifuge Retention Capacity Test

As used herein, the centrifugal retention capacity (CRC) is a measure of the absorbent capacity of the superabsorbent material or fiber after being subjected to centrifugation under controlled conditions. The superabsorbent sample to be tested is taken from superabsorbent material which is prescreened through U.S. standard #30 mesh and retained on U.S. standard #50 mesh to obtain a particle size of between 300 and 600 microns. Testing a fiber sample is performed “as-is” without fractionation. The CRC can be measured by placing 0.200 grams of the sample material to be tested (moisture content of less than 5 weight percent) into a water-permeable bag which will contain the sample while allowing the test solution (0.9 percent by weight sodium chloride solution) to be freely absorbed by the sample. A heat-sealable tea bag material (grade 542, commercially available from Kimberly-Clark Corporation, Neenah, Wis.) works well for most applications. The bag is formed by folding a 12.7 centimeter by 7.62 centimeter sample of the bag material in half and heat sealing two of the open edges to form a 6.35 by 7.62 centimeter rectangular pouch. The heat seals should be about 0.635 centimeters inside the edge of the material. After the sample is placed in the pouch, the remaining open edge of the pouch is also heat-sealed. Empty bags are also made to be tested with the sample bags as controls. Three sample bags are tested for each superabsorbent material. The sealed bags are placed between two Teflon coated fiberglass screens having 0.635 centimeter openings (Taconic Plastics, Inc., Petersburg, N.Y.) and submerged in a pan of 0.9 percent by weight sodium chloride solution at about 23° C., making sure that the screens are held down until the bags are completely wetted. After wetting, the samples remain in the solution for 30 minutes, at which time they are removed from the solution and temporarily laid on a nonabsorbent flat surface. The wet bags are then placed into the basket of a suitable centrifuge capable of subjecting the samples to a force equivalent to 300 times the acceleration of gravity. A suitable centrifuge is a Heraeus Instruments Labofuge 400, having a water collection basket, digital rotations per minute (rpm) gauge, and machined drainage basket adapted to hold and drain the samples. The samples must be placed in opposing positions within the centrifuge to balance the basket when spinning. The bags are centrifuged at a target of 1600 rotations per minute, but within the range of 1500-1900 rotations per minute, for 3 minutes (target force of 300 times the acceleration due to gravity). The bags are removed and weighed, with the empty bags (controls) being weighed first, followed by the bags containing the superabsorbent material or fiber. The amount of fluid absorbed and retained by the superabsorbent material or fiber, taking into account the fluid retained by the bag material alone, is the Centrifugal Retention Capacity of the superabsorbent material or fiber, expressed as grams of fluid per gram of material. This calculation is done by the following equation:

$$CRC = \frac{( {W}_{s} - {W}_{e} - {W}_{d} )}{{W}_{d}}$$

where “CRC” is the Centrifugal Retention Capacity of the sample (grams/gram), “Wₛ” is the after centrifuged mass of the teabag and the sample (grams), “Wₑ” is the average after centrifuged mass of the empty teabag (grams), and “W_{d}” is the dry mass of the sample (grams). The CRC measurements for each of three replicate are averaged to provide the CRC value of the material.

Vortex Time Test

The Vortex Time Test measures the amount of time in seconds required for a predetermined mass of a superabsorbent material to close a vortex created by stirring 50 milliliters of 0.9 by weight sodium chloride solution at 600 revolutions per minute on a magnetic stir plate. The time it takes for the vortex to close is an indication of the free swell absorbing rate of the superabsorbent material. As differences in centrifuge retention capacity (which can be dependant on particle size) between superabsorbent materials can affect the vortex time, the vortex time test can be compensated for better comparison of various superabsorbent materials by adjusting the amount of superabsorbent material added to the 50 milliliter sodium chloride solution as compared to a standard conventional superabsorbent.

The amount of superabsorbent material to be used in the vortex time test is determined by comparison of the centrifuge retention capacity of the new sample against a conventional superabsorbent material, such as FAVOR® 880, available from Stockhausen, Inc., Greensboro, N.C., which has a centrifuge retention capacity value of 33.6 g/g. For determining the vortex time of FAVOR® 880, 2.0 grams of FAVOR® 880 are added to 50 milliliters of 0.9 weight percent sodium chloride solution. The amount of a different superabsorbent material to be used in the vortex time test can be determined by the following formula.

$$C = \frac{2.0 grams \times A}{B}$$

Where “A” is the centrifuge retention capacity of the standard superabsorbent (FAVOR® 880), or 33.6 g/g, “B” is the centrifuge retention capacity of the second superabsorbent material, and “C” is the amount of the second superabsorbent material to be used in the vortex time test.

The vortex time test is preferably done at standard room atmosphere conditions, where the temperature is 23° C.±1° C. and relative humidity is 50 percent ±2 percent. The vortex time test is done by measure 50 milliliters (±0.01 milliliter) of 0.9 by weight sodium chloride solution into the 100 milliliter beaker. Place a 7.9 millimeters×32 millimeters TEFLON® covered magnetic stir bar without rings (such as that commercially available from Baxter Diagnostics, under the trade designation S/P® brand single pack round stirring bars with removable pivot ring) into the beaker. Program a magnetic stir plate (such as that commercially available from PMC Industries, under the trade designation DATAPLATE® Model #721) to 600 revolutions per minute. Place the beaker on the center of the magnetic stir plate such that the magnetic stir bar is activated. The bottom of the vortex should be near the top of the stir bar. The superabsorbent material is pre-screened through U.S. standard #30 mesh and retained on U.S. standard #50 mesh. The superabsorbent material, therefore, has a particle size of between 300 and 600 microns. Weigh out the required mass of the superabsorbent material to be tested on weighing paper. While the sodium chloride solution is being stirred, quickly pour the superabsorbent material to be tested into the saline solution and start a stopwatch. The superabsorbent material to be tested should be added to the saline solution between the center of the vortex and the side of the beaker. Stop the stopwatch when the surface of the saline solution becomes flat and record the time. The time, recorded in seconds, is reported as the vortex time.

Intake Rate and Lock-up Testing

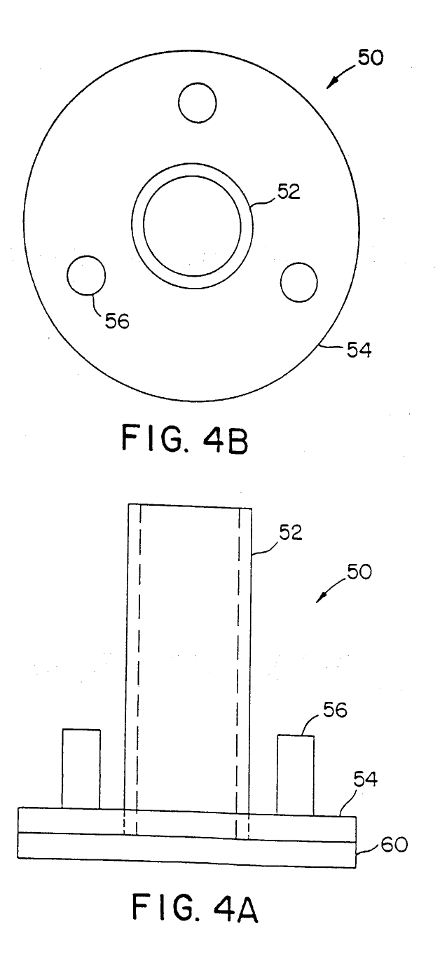

Intake rate is determined by pre-weighing a 7.68 centimeter diameter absorbent composite sample, and placing the 7.68 centimeter diameter sample under a cylindrical port device as shown in FIGS. 4A and 4B. FIG. 4A shows cylindrical port device 50 having cylinder 52 and base 54. Cylindrical port device 50 can be made from various materials, such as plastic, and has a weight that will result in pressure being placed on sample 60 below the cylindrical port device 50. As shown in FIG. 4B, additional weights 56 can be placed on base 54 for testing sample 60 at higher pressures. Sample 60 as a diameter substantially equal to the diameter of base 54, which is 7.68 centimeters for each in the present testing, and is placed under base 54 during testing. For the examples below weights 56 were added to obtain a testing pressure on the samples of about 0.09 pounds per square inch (psi).

Cylinder 52 is hollow with an inner diameter of 2.54 centimeters, allowing for liquid to be poured into cylinder 52 and contact sample 60 below. 15 cubic centimeters (cc) of 0.9% by weight sodium chloride solution is poured into the cylindrical port device. The time required for the volume of liquid to be absorbed into the samples at the base of the device is recorded. Divide the total charge of 15 cubic centimeters by the intake time for each sample to obtain the intake rate for that sample.

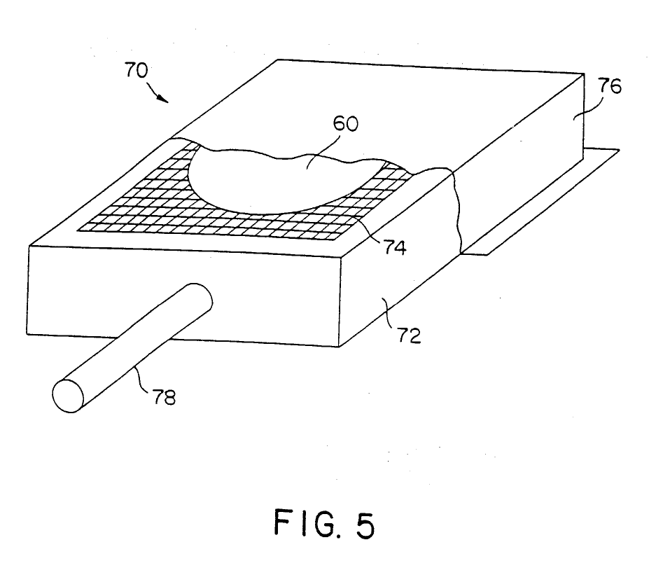

A typical vacuum apparatus useful for lock-up testing is shown in FIG. 5. Vacuum apparatus 70 includes base 72 with mesh screen 74 and vacuum tube 78 attached to a vacuum source (not shown). Sample 60 is placed onto mesh screen 74, typically a #100 mesh screen, and sample 60 and base 72 are covered by gas impervious rubber dam 76. A vacuum is applied through vacuum tube 78 and, because rubber dam creates a seal around base 72, the vacuum pulls an amount of liquid from sample 60. The amount of fluid maintained by sample 60 is determined by subtracting the dry weight of the sample from the wet weight of the sample after application of the vacuum, and converting the net weight to milliliters using the density of the test liquid.

Lock-up testing can be done following the intake rate test by waiting 60 seconds, putting each sample absorbent composite on a vacuum apparatus and applying a vacuum of about −13.5 pounds per square inch gauge (psig) for two minutes. After applying vacuum for 60 seconds, the mass of sodium chloride solution left in the sample is determined. Determine liquid lock-up by dividing the mass of liquid remaining in the sample by the total initial insult.

The intake and lock-up tests are repeated for each sample three times for a total insult of 45 cubic centimeters sodium chloride solution applied to the sample. However, after the lock-up testing of each sample, the sample composite has been drained of some of the liquid from the intake rate testing insult. Therefore, for the second intake and lock-up tests, a new (although nominally same in composition) sample is used. The second, nominally identical sample is given a first 15 cubic centimeter insult of 0.9 percent by weight sodium chloride solution insult (equivalent to the amount from the first intake rate test) and, after waiting 15 minutes, a second 15 cubic centimeter insult of 0.9 percent by weight sodium chloride solution, for a total of 30 cubic centimeters of 0.9 percent by weight sodium chloride solution. After the second intake rate test a second lock-up test is performed as above, so the third intake rate test also uses a new, nominally identical sample. The third, nominally identical sample is given a first 15 cubic centimeter insult of 0.9 percent by weight sodium chloride solution (equivalent to the amount from the first intake rate test), a second of 15 cubic centimeter insult of 0.9% by weight sodium chloride solution after 15 minutes, and, after waiting an additional 15 minutes, a third 15 cubic centimeter insult of 0.9 percent by weight sodium chloride solution is added to the sample for a total of 45 cubic centimeters of 0.9 percent by weight sodium chloride solution.

The calculations for intake rate are the same each time. To calculate lock-up on subsequent insults, divide the cumulative mass of liquid remaining in the sample after vacuum by the cumulative amount of liquid that has been added to the sample. When the intake rate and lock-up tests are complete a saturation test was conducted on a nominally identical sample to determine total saturation capacity of the absorbent composite.

The liquid saturated retention capacity of the composite is determined as follows. The material to be tested is weighed and submerged in an excess quantity of 0.9% by weight sodium chloride solution at standard TAPPI conditions. The material to be tested is allowed to remain submerged for about 20 minutes. After the 20 minute submerging, the material is removed and, referring to FIG. 5, placed on a vacuum apparatus with 0.25 inch diameter openings and covered with #100 mesh screen 74 which, in turn, is connected to vacuum source 78 and covered with a flexible rubber dam material 76. A vacuum of about −13.5 pounds per square inch gauge is drawn on the vacuum apparatus for a period of about 3 minutes with the use of, for example, house vacuum supply. The material being tested is then removed from the apparatus and weighed. The amount of liquid retained by the material being tested is determined by subtracting the dry weight of the material from the wet weight of the material (after application of the vacuum), converting the weight to milliliters by using the density of the test liquid, and is reported as the liquid saturated retention capacity in milliliters of liquid retained. For relative comparisons, the weight of liquid held (wet weight after application of vacuum minus dry weight) can be divided by the weight of the material 60 to give specific liquid saturated retention capacity in grams of liquid retained per gram of tested material.

Because intake rate and liquid lock-up fraction change as a function of saturation, the data should be normalized to a common set of criteria. Using the saturated capacity of the composite, determine the percent saturation of the sample following each insult. For example, a 15 cubic centimeter insult to a sample with a 45 cubic centimeter saturation capacity yields 33% saturation. Plot the intake rate of an absorbent composite as a function of the percent saturation. Interpolate the effective intake rate at the 80% composite saturation level.

To demonstrate the calculations for determining intake rate at 80% composite saturation, the following is the calculation for determining the intake rate of an airformed composite made with DRYTECH® 2035 (reported as Sample 6 below), a superabsorbent material available from Dow Chemical Company, Midland, Mich. The composite was prepared by combining 1.20 grams of DRYTECH® 2035 superabsorbent material with 1.20 grams wood pulp fibers, designated as CARESSA® 1300, available from Buckeye Corporation of Memphis, Tenn., in an airforming handsheet former unit. The airforming handsheet former unit mixed the materials and formed a web of intermingled superabsorbent particles and fibers directly onto a porous sheet of tissue. The tissue was a 9.8 pound White Forming Tissue available from American Tissue Inc., Neenah, Wis. A second layer of tissue was placed above the web following web formation. The airformed composite was 7.68 centimeters in diameter and was compressed to a density of 0.2 grams/cubic centimeter using a Carver Press.

The saturation capacity of the 7.68 centimeter disc composite was determined to be 29.13 cubic centimeters liquid. Intake rate is determined by dividing the insult amount (15 cubic centimeters) by the intake time. Table 1 summarizes the intake time and intake rate results for each of the three insults done on the composite during the intake rate test. A composite saturation percent value is calculated for the composite at each of 15 cubic centimeters, 30 cubic centimeters, and 45 cubic centimeters liquid by dividing the cumulative liquid amount (15, 30, or 45 cc) by the saturation capacity of the composite and multiplying by 100%. The results are summarized in Table 5. The intake rate data are plotted against the respective composite saturation percent in a scatter plot with smoothed lines. FIG. 8 shows the scatter plot for the values of Table 1 as done using the spreadsheet Microsoft EXCEL 97®. To determine the intake rate at 80% composite saturation, a line is drawn along the scatter plot parallel to the y-axis at 80% composite saturation. The intake rate is then determined from the scatter plot at the point where the line intersects the curve.

TABLE 1

| | Insult | Cumulative | Intake | Intake | Composite |
|---|---|---|---|---|---|
| Insult | Amount | Insult Amount | Time | Rate | Saturation |
| # | (cc) | (cc) | (sec) | (cc/sec) | (%) |
| 1 | 15 | 15 | 7.28 | 2.06 | 52 |
| 2 | 15 | 30 | 4.46 | 3.36 | 103 |
| 3 | 15 | 45 | 4.02 | 3.73 | 154 |

Liquid lock-up fractions are also normalized. Rather than normalizing to the composite saturation level, however, the lock-up fractions are normalized to the saturation capacity of the superabsorbent material alone. This is done to better reflect the ability of the superabsorbent material to lock-up liquid relative to the superabsorbent material total saturation capacity. Again, example normalization calculations will be done using the composite having DRYTECH® 2035. The saturation capacity of the superabsorbent material for the composite is necessary for the calculations and was determined by the Centrifuge Retention Capacity Test to be 30 gram/gram. As the matrix fibers of the composite also absorb a small amount of fluid, this absorption will be taken into account in the calculations. The typical intrafiber capacities, as determined by the centrifuge retention capacity test, of CARESSA® 1300 fibers and CR-1654 fibers (available from Bowater Corporation, Coosa Pines, Ala.) are about 1.0 gram liquid/gram fiber.

The lock-up fraction at 50% superabsorbent saturation is determined by plotting the lock-up fraction test data against the superabsorbent material saturation and then interpolating the value from the plot. Lock-up fraction is equal to the amount of liquid in the sample after vacuuming divided by the cumulative insult amount. The results for the composite are summarized in Table 2. Superabsorbent saturation is determined according to the following formula.

$$SuperabsorbentSaturation = \frac{( A ) - ( B ) ( C ) ( D )}{( B ) ( E ) ( F )}$$

Where “A” is the amount of liquid in the composite after vacuum, “B” is the composite dry mass, “C” is the fiber fraction, “D” is the typical intrafiber capacity, “E” is the superabsorbent fraction, and “F” is the centrifuge retention capacity of the superabsorbent. The fiber fraction is the total composite fiber weight divided by the total composite weight. Likewise the superabsorbent fraction is the total superabsorbent material weight divided by the total composite weight. The superabsorbent saturation values for the composite are summarized in Table 2.

TABLE 2

| | | | Amount of | | | |
|---|---|---|---|---|---|---|
| | | | Liquid in | | | |
| | | Cumulative | Composite | Composite | SAM | |
| | Insult | Insult | after | Dry | Sat- | |
| Insult | Amount | Amount | Vacuum | Mass | uration | Lock-up |
| # | (cc) | (cc) | (cc) | (g) | (%) | Fraction |
| 1 | 15 | 15 | 8.90 | 2.51 | 20 | 0.59 |
| 2 | 15 | 30 | 19.57 | 2.49 | 49 | 0.65 |
| 3 | 15 | 45 | 26.58 | 2.50 | 68 | 0.59 |

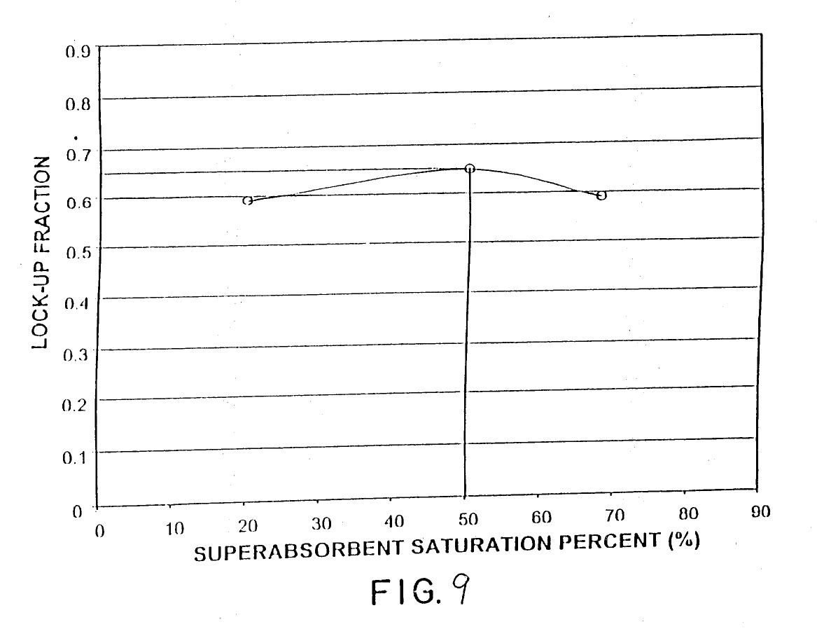

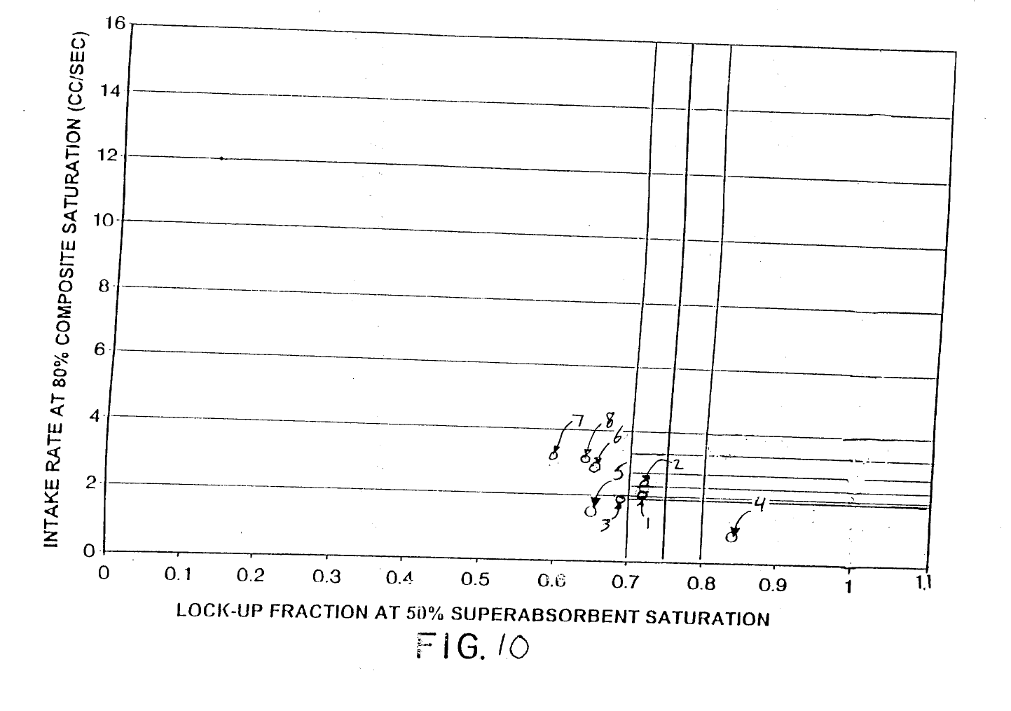

The lock-up fraction data are plotted against the respective superabsorbent material saturation percent in a scatter plot with smoothed lines. FIG. 9 shows the scatter plot for the values of Table 2 as done using the spreadsheet Microsoft EXCEL 97®. To determine the lock-up fraction at 50% superabsorbent saturation, a line is drawn across the scatter plot parallel to the y-axis at 50% superabsorbent saturation. The lock-up fraction is then determined from the scatter plot at the point where the line intersects the curve.

### EXAMPLES

The stiffness index and the vortex time of several commercial superabsorbent materials were tested according to the tests described above as a comparison to the superabsorbent materials of this invention. The results of the testing are reported in Table 3. The unmodified superabsorbent materials tested were HYSORB® 7050, available from BASF, Portsmouth, Va., DRYTECH® 2035, available from Dow Chemical Company, Midland, Mich., and FAVOR® 880 and FAVOR® 9543, both available from Stockhausen, Inc., Greensboro, N.C.

TABLE 3

| Superabsorbent Material | Stiffness Index | Vortex Time (seconds) |
|---|---|---|
| HYSORB ® 7050 | 0.73 | 72 |
| DRYTECH ® 2035 | 0.46 | 116 |
| FAVOR ® 880 | 0.65 | 98 |
| FAVOR ® 9543 | 0.92 | 83 |

To demonstrate the absorbent composites of this invention, two airformed composites, Samples 1 and 2, were made including modified FAVOR® SXM 9543 superabsorbent material. The FAVOR® SXM 9543 was modified by freeze-drying according to the following method. An amount of distilled water was added into a one gallon HOBART® mixer (Model N50, manufactured by Hobart Canada, Ontario, Canada). 100 grams of FAVOR® SXM 9543 superabsorbent particles were added into the mixer while the stirrer was on. After stirring for about 2 minutes, the swollen superabsorbent particles were discharged into a pan (10 inches by 20 inches or 25.4 centimeters by 50.8 centimeters) to form a uniform thin layer. The pan was placed into a VirTis Genesis freeze dryer (Model 25 EL) available from The VirTis Inc. of Gardiner, N.Y. The superabsorbent material was freeze-dried in the freeze dryer at a shelf temperature of less than −50° C., a condenser temperature of less than −70° C., and a vacuum of less than 100 millitorrs. A portion of the freeze-dried superabsorbent particles was sieved to obtain a size range of about 300 to 600 micron particles which were used to test the stiffness index and the vortex time of the modified superabsorbent particles. Non-sieved, as-prepared freeze-dried superabsorbent particles were used to make the absorbent composites of Samples 1 and 2. The modified freeze-dried superabsorbent particles of Sample 1 were made as described above by absorbing 200 grams of distilled water into 100 grams of FAVOR® SXM 9543 (swelling level of 2 grams/gram) and freeze-drying, and the superabsorbent particles had a stiffness index of 0.93 and vortex time of 28.5 seconds. The modified freeze-dried superabsorbent particles of Sample 2 were made as described above by absorbing 500 grams of distilled water into 100 grams of FAVOR® SXM 9543 (swelling level of 5 grams/gram) and freeze-drying, and the superabsorbent particles had a stiffness indexes of 0.87 and vortex time of 16.7 seconds. The absorbency under load, stiffness index and vortex time, as determined by the tests and calculations described above, for each of the freeze-dried superabsorbent FAVOR® SXM 9543 of Samples 1 and 2, as well as the unmodified FAVOR® SXM 9543 for comparison, are summarized in Table 4.

TABLE 4

| | AUL | Stiffness | Vortex Time |
|---|---|---|---|
| Superabsorbent Material | (g/g) | Index | (seconds) |
| Freeze-dried FAVOR ® SXM 9543 | 18.9 | 0.93 | 28.5 |
| (Samples 1 and 7) (2 g/g) | | | |
| Freeze-dried FAVOR ® SXM 9543 | 17.7 | 0.88 | 16.7 |
| (Samples 2 and 8) (5 g/g) | | | |
| FAVOR ® SXM 9543 | 21.4 | 0.92 | 83 |

Sample 1 was an airformed composite according to this invention prepared by combining 1.20 grams of the appropriate modified freeze-dried FAVOR® SXM 9543 superabsorbent material with 1.20 grams wood pulp fibers, designated as CR-1654, available from Bowater Corporation, Coosa Pines, Ala., in an airforming handsheet former unit. The airforming handsheet former unit mixed the materials and formed a web of intermingled superabsorbent particles and fibers directly onto a porous sheet of tissue. The tissue was a 9.8 pound White Forming Tissue available from American Tissue Inc. A second layer of tissue was placed above the web following web formation. The airformed composite of Sample 1 was 7.68 centimeters in diameter and was compressed to a density of 0.2 gram/cubic centimeter using a Carver Press.

Sample 2, also an airformed composite of this invention, was prepared by combining 1.20 grams of the appropriate modified freeze-dried FAVOR® SXM 9543 with 1.20 grams wood pulp fibers, designated as CR-1654, available from Bowater Corporation, Coosa Pines, Ala., in an airforming handsheet former unit. The airforming handsheet former unit mixed the materials and formed a web of intermingled superabsorbent particles and fibers directly onto a porous sheet of tissue. The tissue was a 9.8 pound White Forming Tissue available from American Tissue Inc. A second layer of tissue was placed above the web following web formation. The airformed composite of Sample 2 was 7.68 centimeters in diameter and was compressed to a density of 0.2 grams/cubic centimeter using a Carver Press.

A third airformed composite, Sample 3, was also prepared as a control comparison to Samples 1 and 2 and using unmodified FAVOR® SXM 9543. Sample 3 was prepared by combining 1.20 grams of FAVOR® SXM 9543 superabsorbent particles with 1.20 grams wood pulp fibers, designated as CR-1654, in an airforming handsheet former unit. The airforming handsheet former unit mixed the materials and formed a web of intermingled superabsorbent particles and fibers directly onto a porous sheet of tissue. The tissue was a 9.8 pound White Forming Tissue available from American Tissue Inc., Neenah, Wisconsin. A second layer of tissue was placed above the web following web formation. The airformed composite of Sample 3 was 7.68 centimeters in diameter and was compressed to a density of 0.2 grams/cubic centimeter using a Carver Press.

Two commercially available diapers, HUGGIES® Supreme Step-3 (Bag Code NM034102b0545-1900), designated Sample 4, and PAMPERS® Premium Size-2 (Bag Code 1121U017261559), designated Sample 5 were also tested for comparison. To obtain samples for testing, the diaper was placed on a die cutting device and a 7.68 centimeter diameter sample was taken from the target area of the diaper. The centerpoint of the 7.68 centimeter die cut sample was 16.64 centimeters from the front end of the absorbent pad and spaced in the middle of the absorbent pad in the cross-direction. Following punching of the sample, all layers were removed leaving only the superabsorbent-fluff layer of the products.

To separate the superabsorbent material from the fluff of a diaper for CRC testing, the absorbent core of the diaper is first placed in an airlaid handsheet former. The handsheet former takes the relatively dense absorbent core of the product and forms a bulky handsheet on a sheet of tissue. This step in the procedure opens the superabsorbent material/fluff matrix before being placed in a “diaper destroyer” for separation.

The handsheet former has an upper cylinder shaped compartment which is separated from a rectangular lower compartment by a diffusion screen which helps ensure uniform handsheet formation. The absorbent core is separated from the product over the upper compartment of the airlaid handsheet former so that any superabsorbent material or fluff that falls out during removal of the absorbent core goes into the upper compartment. The outer cover, body-side liner, and any surge materials and/or barrier tissues are scraped with a spatula to remove any remaining superabsorbent material or fluff.

The lid is placed on the upper compartment of the handsheet former, and pulsating air in the upper compartment and a vacuum at the bottom of the lower compartment are turned on. The components of the absorbent core are formed into a bulky superabsorbent material/fluff airlaid handsheet on a sheet of tissue located at the bottom of the lower compartment. The tissue provides a barrier to the superabsorbent material and fluff. The handsheet former is operated until no superabsorbent material and approximately 0.5 grams or less of fluff is visible in the upper compartment.

The bulky superabsorbent/fluff pad is then carefully placed in the diaper destroyer. The mechanics of the diaper destroyer are the reverse of the handsheet former. Pulsating air circulating at the bottom of the diaper destroyer breaks apart the bulky pad. The superabsorbent material from the pad collects at the bottom, whereas the fluff is drawn off the top with a vacuum. When most of the fluff has been drawn off, the diaper destroyer is turned off and the superabsorbent material is collected on a nonstick metal tray. Shaking the superabsorbent material on the tray tends to clump the remaining fluff fibers together for easier removal with tweezers, thus avoiding contamination of the superabsorbent material with moisture, etc. from the operator's hands. The collected superabsorbent material is placed in a labeled glass bottle for further characterization.

An additional airformed composite, Sample 6 (described above in the calculations for intake time and lock-up fraction), was made with DRYTECH® 2035. The composite was prepared by combining 1.20 grams of DRYTECH® 2035 superabsorbent material with 1.20 grams wood pulp fibers, designated as CARESSA® 1300, in an airforming handsheet former unit. The airforming handsheet former unit mixed the materials and formed a web of intermingled superabsorbent particles and fibers directly onto a porous sheet of tissue. The tissue was a 9.8 pound White Forming Tissue available from American Tissue Inc., Neenah, Wis. A second layer of tissue was placed above the web following web formation. The airformed composite was 7.68 centimeters in diameter and was compressed to a density of 0.2 grams/cubic centimeter using a Carver Press.

Sample 7 was prepared by combining 1.20 grams of the appropriate modified freeze dried FAVOR® SXM 9543 superabsorbent material with 1.20 grams wood pulp fibers, designated as CARESSA® 1300, in an airforming handsheet former unit. The airforming handsheet former unit mixed the materials and formed a web of intermingled superabsorbent particles and fibers directly onto a porous sheet of tissue. The tissue was a 9.8 pound White Forming Tissue available from American Tissue Inc. A second layer of tissue was placed above the web following web formation. The airformed composite of Sample 7 was 7.68 centimeter in diameter and was compressed to a density of 0.2 gram/cubic centimeter using a Carver Press.

Sample 8 was prepared by combining 1.20 grams of the appropriate modified freeze dried FAVOR® SXM 9543 with 1.20 grams wood pulp fibers, designated as CARESSA® 1300, in an airforming handsheet former unit. The airforming handsheet former unit mixed the materials and formed a web of intermingled superabsorbent particles and fibers directly onto a porous sheet of tissue. The tissue was a 9.8 pound White Forming Tissue available from American Tissue Inc. A second layer of tissue was placed above the web following web formation. The airformed composite of Sample 8 was 7.68 centimeter in diameter and was compressed to a density of 0.2 grams/cubic centimeter using a Carver Press.

The centrifuge retention capacities (in gram of fluid per gram of superabsorbent material) of the superabsorbent materials of Samples 1-8 were determined according to the Centrifuge Retention Capacity Test described above and are summarized in Table 5.

TABLE 5

| | Superabsorbent Material | Centrifuge Retention |
|---|---|---|
| Sample(s) | (SAM) | Capacity (g/g) |
| 1 and 7 | Freeze-dried FAVOR ® | 20.3 |
| | SXM 9543 (2 g/g) | |
| 2 and 8 | Freeze-dried FAVOR ® | 20.2 |
| | SXM 9543 (5 g/g) | |
| 3 | FAVOR ® SXM 9543 | 23.2 |
| 4 | SAM of HUGGIES ® | 30 |
| 5 | SAM of PAMPERS ® | 26 |
| 6 | DRYTECH ® 2035 | 30 |

Samples 1-8 were tested by the Intake Rate and Liquid Lock-up Tests described above. The results of testing three replicate samples are averaged and summarized in Table 6. FIG. 10 is graph of the intake rates plotted against the lock-up fractions of the samples listed in Table 5. Samples 1 and 2 had both desirable intake rate and lock-up characteristics. Intake rate and lock-up fraction can be dependent on the structure of the composite as well as the specific superabsorbent material and fiber combination.

Sample 3 did demonstrate a desirable intake rate but also a less than desirable lock-up fraction. Sample 4, the HUGGIES® diaper sample, exhibited a desirable liquid lock-up but a less than desirable intake rate of this invention. Sample 5, the PAMPERS® diaper sample, exhibited less than both the desirable intake rate and liquid lock-up fraction of this invention. Samples 7 and 8 also did not have the desired lock-up fraction and demonstrate that the lock-up is not dependent on the superabsorbent material alone, but on the specific superabsorbent material and fibers used, as well as the structure of the absorbent composites. Table 6 shows the superabsorbent material of the samples as a percentage of the weight of fiber/superabsorbent material. The liquid lock-up numbers in Table 6 are at a 50% superabsorbent material (SAM) saturation and the intake rates are at 80% absorbent composite saturation.

TABLE 6

| | | | | Intake Rate |
|---|---|---|---|---|
| | | Superabsorbent | Liquid | (cc/s) at 80% |
| | | Material | Lock-up at 50% | composite |
| Sample | Structure | (wt. %) | SAM saturation | saturation |
| 1 | Airformed | 50 | 0.72 | 2.1 |
| | Composite | | | |
| 2 | Airformed | 50 | 0.72 | 2.4 |
| | Composite | | | |
| 3 | Airformed | 50 | 0.69 | 1.9 |
| | Composite | | | |
| 4 | Diaper core | 42 | 0.84 | 0.80 |
| 5 | Diaper core | 59 | 0.65 | 1.7 |
| 6 | Airformed | 50 | 0.65 | 2.8 |
| | Composite | | | |
| 7 | Airformed | 50 | 0.60 | 3.3 |
| | Composite | | | |
| 8 | Airformed | 50 | 0.64 | 3.2 |
| | Composite | | | |

Various conventional techniques may be employed to determine the quantitative amount of superabsorbent material within a test sample. Suitable analytical techniques include, for example, a sulfated ash measurement method, such as described in “Vogel's Textbook of Quantitative Inorganic Analysis,” Fourth Edition, revised by J. Bassett, R. C. Denney, G. H. Jeffery, J. Mendham, Longman Inc., 1973, pp. 479-481, herein incorporated by reference. Another suitable technique would be an ion exchange method (e.g. sodium ion exchange), such as described in “Treatise on Analytical Chemistry,” Volume 1, edited by I. M. Kolthoff and Phillip J. Elving, Interscience Publishers, Inc., 1961, pp. 345-350, herein incorporated by reference. Another suitable technique includes atomic absorption methods, such as described in “Vogel's Textbook of Quantitative Inorganic Analysis,” Fourth Edition, revised by J. Bassett, R. C. Denney, G. H. Jeffery, J. Mendham, Longman Inc., 1978, pp. 310-845, herein incorporated by reference. “The Encyclopedia of Industrial Chemical Analysis,” Volume 18, edited by Foster Dee Snell and Leslie S. Ettre, Interscience Publishers, Inc., 1973, at pp. 207-259, describes well known, conventional techniques for quantitatively measuring the amount of sodium within a sample, herein incorporated by reference.

The amount of superabsorbent material present in each of Samples 4 and 5 was determined by sulfated ash testing. The sulfated ash procedure converts the sodium or other cations carboxyl salt polymers, such as polyacrylate or carboxymethyl cellulose superabsorbent material, to the corresponding sulfate salt. The sulfate salt is determined gravimetrically and is calculated to the weight of the carboxyl salt polymer by applying a standard factor determined from a sample of the pure polymer. The sample is charred over a low flame to remove the bulk of the volatile matter, cooled, moistened with 1:1 sulfuric acid, the excess acid volatilized, and the ashing completed as in a regular ash determination.

The sulfated ash method can be applied to a wide range of sample sizes, including a whole diaper. Weigh a sample to the nearest 0.001 gram, into a previously ignited and tarred (to the nearest 0.1 milligram) porcelain dish or crucible. When determining the superabsorbent material content of whole diapers, as much as possible of extraneous product components (e.g. tapes, elastics) should be trimmed off first, but not so much as to lose any superabsorbent material granules. Record both the whole product weight and the trimmed weight.

Ignite the sample over a burner flame until most of the carbonaceous materials are burned off. Cool, and then moisten the entire residue with 1:1 sulfuric acid. Slowly evaporate the excess acid over a low flame so as to avoid spattering. Complete the ignition by placing the sample in a muffle, or alternatively use a forced air Meker-type burner, at 800° C.-850° C. for 60 minutes or until the ash is free of carbon. Cool in a desiccator and weigh to the nearest 0.1 mg.

A “standard factor” is then determined for the sample. The standard factor is determined by the following formula.

$$StandardFactor ( F ) = \frac{( gramsofovendrypolymer )}{( gramssulfatedash ) ( 0.95 )}$$

Dividing the standard factor by 0.95 takes into account the absorption of moisture that increases the weight of the sample. Depending on humidity and exposure conditions, the superabsorbent material can absorb significant levels of water (e.g. 59% at 80% RH, 100° F.). A standard 5% moisture basis is typically used in the calculation as an estimate of the additional moisture weight absorbed by the sample.

The presence of any other inorganic compound or cation, besides the superabsorbent material, will generally give a positive interference. The absence of interferences must be known and/or blank corrections must be determined if accurate results are to be obtained by this method. If samples of the material or product without added superabsorbent material are available, these can be carried through the procedure determine a correction factor. If the individual product components are available, they can likewise be analyzed and a correction factor calculated as a weighted average. The correction factor is then calculated by dividing the grams of sulfated ash by the grams of the equivalent superabsorbent free sample or components. For samples unable to have a correction factor determined by these methods, an averaged correction factor can be determined on virgin wood fluff. The typically used correction factor is 0.00513.

The calculation for the percent carboxyl salt polymer by weight of the sample is calculated by the following formula.

$$\% CarboxylSaltPolymer = \frac{( A - BC ) ( F ) ( 100 )}{( C )}$$

Where “A” is the weight of sulfated ash from the sample, “B” is the correction factor, “C” is the original weight of sample, and “F” is the standard factor. The superabsorbent material obtained by the sulphated ash testing is assumed to be at a 5% moisture basis.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

What is claimed is:

1. An absorbent composite, comprising:
water-insoluble fibers; and
a superabsorbent material, the superabsorbent material having a stiffness index of greater than about 0.87 and a vortex time of less than about 40 seconds.

2. The absorbent composite of claim 1, wherein the superabsorbent material has a vortex time of less than about 30 seconds.

3. The absorbent composite of claim 2, wherein the superabsorbent material has a vortex time of less than about 20 seconds.

4. The absorbent composite of claim 1, wherein the superabsorbent material has a stiffness index of greater than about 0.92.

5. The absorbent composite of claim 4, wherein the superabsorbent material has a vortex time of less than about 30 seconds.

6. The absorbent composite of claim 5, wherein the superabsorbent material has a vortex time of less than about 20 seconds.

7. The absorbent composite of claim 4, wherein the superabsorbent material has a stiffness index of greater than about 1.0.

8. The absorbent composite of claim 7, wherein the superabsorbent material has a vortex time of less than about 30 seconds.

9. The absorbent composite of claim 8, wherein the superabsorbent material has a vortex time of less than about 20 seconds.

10. The absorbent composite of claim 1, wherein the water-insoluble fibers comprise hydrophilic fibers.

11. The absorbent composite of claim 10, wherein the water-insoluble fibers comprise hydrophilically treated hydrophobic fibers.

12. The absorbent composite of claim 1, wherein the superabsorbent material includes a polymer selected from the group consisting of an anionic polymer, a cationic polymer, an acidic polymer, a basic polymer, and combinations thereof.

13. The absorbent composite of claim 1, wherein the superabsorbent material includes a polymer selected from the group consisting of sodium-polyacrylate, polyvinyl amines, and combinations thereof.

14. The absorbent composite of claim 1, wherein the absorbent composite comprises the superabsorbent material in a weight amount of about 10 to 70 weight percent based on total weight of the absorbent composite.

15. The absorbent composite of claim 14, wherein the absorbent composite comprises a water-insoluble fiber in a weight amount of about 30 to 90 weight percent, based on total weight of the absorbent composite.

16. The absorbent composite of claim 15, wherein the absorbent composite further comprises a binder in a weight amount of about 0 to 20 weight percent, based on total weight of the absorbent composite.

17. The absorbent composite of claim 1, wherein the absorbent composite comprises an airformed absorbent composite.

18. The absorbent composite of claim 1, wherein the absorbent composite comprises a wetformed absorbent composite.

19. The absorbent composite of claim 1, wherein the absorbent composite comprises a freeze-dried composite.

20. An absorbent article comprising
a liquid-permeable body-side liner;
an absorbent composite, including water-insoluble fibers and a superabsorbent material, the superabsorbent material having a stiffness index of greater than about 0.87 and a vortex time of less than about 40 seconds; and
a substantially liquid-impermeable outer cover adjacent to the absorbent composite material.

21. The absorbent composite of claim 20, wherein the superabsorbent material has vortex time of less than about 30 seconds.

22. The absorbent composite of claim 21, wherein the superabsorbent material has vortex time of less than about 20 seconds.

23. The absorbent composite of claim 20, wherein the superabsorbent material has a stiffness index of greater than about 0.92.

24. The absorbent composite of claim 23, wherein the superabsorbent material has vortex time of less than about 30 seconds.

25. The absorbent composite of claim 24, wherein the superabsorbent material has vortex time of less than about 20 seconds.

26. The absorbent composite of claim 23, wherein the superabsorbent material has a stiffness index of greater than about 1.0.

27. The absorbent composite of claim 26, wherein the superabsorbent material has vortex time of less than about 30 seconds.

28. The absorbent composite of claim 27, wherein the superabsorbent material has vortex time of less than about 20 seconds.

29. The absorbent article of claim 20, wherein the superabsorbent material includes a polymer selected from the group consisting of sodium-polyacrylate, polyvinyl amines, and combinations thereof.

30. The absorbent article of claim 20, wherein the absorbent composite comprises a freeze-dried fibrous composite.

31. The absorbent article of claim 20, wherein the absorbent composite comprises an airformed absorbent composite.

32. The absorbent article of claim 20, wherein the absorbent composite comprises a wetformed absorbent composite.

33. The absorbent article of claim 20, wherein the absorbent composite comprises a non-fibrous absorbent composite.

34. The absorbent article of claim 20, wherein the absorbent article is selected from the group consisting of a diaper, a training pant, a swim wear garment, an adult incontinence garment, a feminine hygiene product, a medical absorbent product.

35. The absorbent article of claim 20, wherein the absorbent composite has an intake rate of at least about 1.9 cubic centimeters of 0.9% by weight sodium chloride aqueous solution per second at 80% absorbent composite saturation and a liquid lock-up fraction of at least about 0.70 at 50% superabsorbent material saturation determined using 0.9% by weight sodium chloride aqueous solution.

36. An absorbent composite, comprising:
water-insoluble fibers; and
a superabsorbent material, the superabsorbent material having a stiffness index of greater than about 0.87 and a vortex time of less than about 40 seconds;
wherein the absorbent composite has an intake rate of at least about 1.9 cubic centimeters of 0.9% by weight sodium chloride aqueous solution per second at 80% absorbent composite saturation and a liquid lock-up fraction of at least about 0.70 at 50% superabsorbent material saturation determined using 0.9% by weight sodium chloride aqueous solution.

37. The absorbent composite of claim 36, wherein the absorbent composite has an intake rate of at least about 1.9 cubic centimeters of 0.9% by weight sodium chloride aqueous solution per second at 80% absorbent composite saturation and a liquid lock-up fraction of at least about 0.75 at 50% superabsorbent material saturation determined using 0.9% by weight sodium chloride aqueous solution.

38. The absorbent composite of claim 37, wherein the absorbent composite has an intake rate of at least about 1.9 cubic centimeters of 0.9% by weight sodium chloride aqueous solution per second at 80% absorbent composite saturation and a liquid lock-up fraction of at least about 0.80 at 50% superabsorbent material saturation determined using 0.9% by weight sodium chloride aqueous solution.

39. The absorbent composite of claim 36, wherein the absorbent composite has an intake rate of at least about 2.3 cubic centimeters of 0.9% by weight sodium chloride aqueous solution per second at 80% absorbent composite saturation and a liquid lock-up fraction of at least about 0.70 at 50% superabsorbent material saturation determined using 0.9% by weight sodium chloride aqueous solution.

40. The absorbent composite of claim 35, wherein the superabsorbent material has a vortex time of less than about 30 seconds.

41. The absorbent composite of claim 40, wherein the superabsorbent material has a vortex time of less than about 20 seconds.

42. The absorbent composite of claim 36, wherein the superabsorbent material has a stiffness index of greater than about 0.92.

43. The absorbent composite of claim 42, wherein the superabsorbent material has a vortex time of less than about 30 seconds.

44. The absorbent composite of claim 42, wherein the superabsorbent material has a vortex time of less than about 20 seconds.

45. The absorbent composite of claim 42, wherein the superabsorbent material has a stiffness index of greater than about 1.0.

46. The absorbent composite of claim 46, wherein the superabsorbent material has a vortex time of less than about 30 seconds.

47. The absorbent composite of claim 46, wherein the superabsorbent material has a vortex time of less than about 20 seconds.

48. The absorbent composite of claim 36, wherein the water-insoluble fibers comprise hydrophilic fibers.

49. The absorbent composite of claim 36, wherein the superabsorbent material includes a polymer selected from the group consisting of sodium-polyacrylate, polyvinyl amines, and combinations thereof

50. The absorbent composite of claim 37, wherein the absorbent composite comprises the superabsorbent material in a weight amount of about 10 to 70 weight percent based on total weight of the absorbent composite.

51. The absorbent composite of claim 50, wherein the absorbent composite comprises the fibers in a weight amount of about 30 to 60 weight percent based on total weight of the absorbent composite.

52. The absorbent composite of claim 51, further comprising a binder material in a weight amount of about 1 to 20 weight percent based on total weight of the absorbent composite

53. The absorbent composite of claim 36, wherein the absorbent composite comprises an airformed absorbent composite.

54. The absorbent composite of claim 36, wherein the absorbent composite comprises a wetformed absorbent composite.

55. The absorbent composite of claim 36, wherein the absorbent composite comprises a freeze-dried composite.

56. An absorbent composite comprising a superabsorbent material and a non-fibrous structure, the superabsorbent material having a stiffness index of greater than about 0.87 and a vortex time of less than about 40 seconds.

57. The absorbent composite of claim 56, wherein the superabsorbent material has a vortex time of less than about 30 seconds.

58. The absorbent composite of claim 57, wherein the superabsorbent material has a vortex time of less than about 20 seconds.

59. The absorbent composite of claim 56, wherein the superabsorbent material has a stiffness index of greater than about 0.92.

60. The absorbent composite of claim 59, wherein the superabsorbent material has a vortex time of less than about 30 seconds.

61. The absorbent composite of claim 60, wherein the superabsorbent material has a vortex time of less than about 20 seconds.

62. The absorbent composite of claim 59, wherein the superabsorbent material has a stiffness index of greater than about 1.0.

63. The absorbent composite of claim 62, wherein the superabsorbent material has a vortex time of less than about 30 seconds.

64. The absorbent composite of claim 63, wherein the superabsorbent material has a vortex time of less than about 20 seconds.

65. The absorbent composite of claim 56, wherein the absorbent composite has an intake rate of at least about 1.9 cubic centimeters of 0.9% by weight sodium chloride aqueous solution per second at 80% absorbent composite saturation and a liquid lock-up fraction of at least about 0.70 at 50% superabsorbent material saturation determined using 0.9% by weight sodium chloride aqueous solution.

66. The absorbent composite of claim 65, wherein the absorbent composite has an intake rate of at least about 1.9 cubic centimeters of 0.9% by weight sodium chloride aqueous solution per second at 80% absorbent composite saturation and a liquid lock-up fraction of at least about 0.75 at 50% superabsorbent material saturation determined using 0.9% by weight sodium chloride aqueous solution.

67. The absorbent composite of claim 66, wherein the absorbent composite has an intake rate of at least about 1.9 cubic centimeters of 0.9% by weight sodium chloride aqueous solution per second at 80% absorbent composite saturation and a liquid lock-up fraction of at least about 0.80 at 50% superabsorbent material saturation determined using 0.9% by weight sodium chloride aqueous solution.

68. The absorbent composite of claim 65, wherein the absorbent composite has an intake rate of at least about 2.3 cubic centimeters of 0.9% by weight sodium chloride aqueous solution per second at 80% absorbent composite saturation and a liquid lock-up fraction of at least about 0.70 at 50% superabsorbent material saturation determined using 0.9% by weight sodium chloride aqueous solution.
